(19) United States
(12) Patent Application Publication
(10) Pub. No.: US 2017/0086738 A1
(43) Pub. Date: Mar. 30, 2017

(54) BIOLOGICAL SENSOR CONTROL DEVICE, OPERATION METHOD AND OPERATION PROGRAM THEREOF, AND BIOLOGICAL SENSOR SYSTEM

(71) Applicant: FUJIFILM Corporation, Tokyo (JP)

(72) Inventor: Yuya KUDO, Tokyo (JP)

(73) Assignee: FUJIFILM Corporation, Tokyo (JP)

(21) Appl. No.: 15/259,646

(22) Filed: Sep. 8, 2016

(30) Foreign Application Priority Data
Sep. 29, 2015 (JP) 2015-191367

(51) Int. Cl. A61B 5/00; A61B 5/0205

(52) U.S. Cl. CPC A61B 5/6801; A61B 5/0022; A61B 5/0024; A61B 5/7282; A61B 5/02055; A61B 5/0402

(57) ABSTRACT

There are provided a biological sensor control device, an operation method and operation program thereof, and a biological sensor system capable of effectively using the power of a battery of a wearable biological sensor device within a measurement period. In a case where the measurement period of a wearable biological sensor device is shortened, a determination unit changes the driving conditions so as to increase the driving power of the wearable biological sensor device by increasing the number of measurement items and/or by shortening the measurement interval. The determination unit determines the driving conditions in which the remaining capacity becomes a preset value or less at the end of the measurement period.

### CROSS-REFERENCE TO RELATED APPLICATIONS

The present application claims priority under 35 U.S.C. §119 to Japanese Patent Application No. 2015-191367, filed on Sep. 29, 2015, all of which are hereby expressly incorporated by reference into the present application.

### BACKGROUND OF THE INVENTION

1. Field of the Invention

The present invention relates to a biological sensor control device, an operation method and non-transitory computer readable recording medium storing an operation program thereof, and a biological sensor system.

2. Description of the Related Art

In the medical field, a technique of attaching a biological sensor device to a patient so that a medical staff member, such as a nurse or a doctor, monitors the condition of the patient has been used. The biological sensor device includes sensors for measuring a plurality of measurement items regarding biological information, such as a heart rate, a respiratory rate, and the amount of body movement. In a case where a patient shows the symptoms of a certain disease during the measurement period, the doctor determines the disease of the patient taking the measurement values of the plurality of measurement items from the biological sensor device into consideration in a comprehensive manner.

A biological sensor device attached to a patient (hereinafter, referred to as a wearable biological sensor device) is supplied with power from the built-in battery, unlike a stationary biological sensor device to which power from a commercial power supply is supplied. Therefore, in the case of using a wearable biological sensor device, it is necessary to appropriately manage the remaining capacity of the battery.

JP2015-123300A discloses a biological sensor control device (referred to as a biological information measurement device) that controls a wearable biological sensor device. The biological sensor control device disclosed in JP2015-123300A sets a priority for a plurality of measurement items, and acquires the remaining capacity of the battery from the wearable biological sensor device. Then, the driving conditions of the wearable biological sensor device are determined based on the priority and the remaining capacity.

More specifically, in a case where the remaining capacity of the battery is equal to or greater than a set value, all of the sensors are driven in a normal power consumption mode regardless of the priority. In a case where the remaining capacity of the battery is less than the set value, a sensor for measuring a low-priority measurement item is driven in a low power consumption mode or is turned off. The low power consumption mode is a mode in which the number of measurements per unit time for each measurement item is less than that in the normal power consumption mode (mode in which the measurement interval for each measurement item is longer than that in the normal power consumption mode). In a case where the remaining capacity of the battery is low, the number of measurement items for which measurement is to be performed is reduced or the measurement interval is increased in order to reduce the power consumption. Accordingly, it is possible to increase the driving time of the wearable biological sensor device.

### SUMMARY OF THE INVENTION

In the case of using a wearable biological sensor device, for example, a measurement period from a current examination date to the next examination date is set by a doctor. In a case where the measurement period is set in this manner, it is ideal that the power of the battery is exhausted exactly at the end of the measurement period from the point of view of effective use of the power of the battery within the measurement period.

In the biological sensor control device disclosed in JP2015-123300A, the driving conditions of the biological sensor device are determined based on the priority and the remaining capacity. However, the measurement period is not taken into consideration. For this reason, a situation may occur in which the power of the battery is exhausted before the end of the measurement period so that it is not possible to perform measurement or the remaining capacity of the battery is relatively large at the end of the measurement period.

In a case where the power of the battery is exhausted before the end of the measurement period so that it is not possible to perform measurement, it may be difficult to determine a disease since the information of measurement values required for the determination of the disease may not be sufficient. On the other hand, in a case where the remaining capacity of the battery is relatively large at the end of the measurement period, an opportunity to acquire more detailed biological information helpful to the determination of a disease by increasing the number of measurement items, for which measurement is to be performed, or by shortening the measurement interval is missed even though there is a capacity for this. In addition, considering that a disposable battery, such as a button battery, is used as a battery of the biological sensor device, a battery with a halfway remaining capacity cannot be reused easily for the next measurement since there is a risk that the battery with a halfway remaining capacity cannot be used any more before the end of the measurement period. Eventually, there is no choice but to throw away the battery with a halfway remaining capacity. This is a waste of resources. Thus, in the technique disclosed in JP2015-123300A, the power of the battery of the wearable biological sensor device was not able to be effectively used within the measurement period.

It is an object of the invention to provide a biological sensor control device, an operation method and non-transitory computer readable recording medium storing an operation program thereof, and a biological sensor system capable of effectively using the power of a battery of a wearable biological sensor device within a measurement period.

In order to solve the aforementioned problem, a biological sensor control device of the invention is a biological sensor control device for controlling a wearable biological sensor device that is attached to a patient and that performs measurement for measurement items regarding biological information of the patient with power supplied from a built-in battery. The biological sensor control device comprises: a first acquisition unit that acquires a remaining capacity of the battery; a second acquisition unit that acquires a measurement period of the wearable biological sensor device; and a determination unit that determines driving conditions of the wearable biological sensor device based on the remaining capacity and the measurement period.

It is preferable that the determination unit determines the driving conditions such that the remaining capacity becomes a preset value or less at the end of the measurement period.

It is preferable that the determination unit determines whether or not to change the driving conditions during the measurement period. More specifically, it is preferable that the determination unit monitors a change in the measurement period and a prediction and measurement difference, which is a difference between a predicted value and a measured value of the remaining capacity, during the measurement period and determines whether or not to change the driving conditions based on monitoring results.

It is preferable that the determination unit determines that the driving conditions are to be changed in a case where the measurement period has been changed or in a case where the prediction and measurement difference is outside a predetermined range.

It is preferable that the determination unit changes the driving conditions so as to increase driving power of the wearable biological sensor device in a case where the measurement period is shortened or in a case where the measured value exceeds the predicted value.

It is preferable that the wearable biological sensor device performs measurement for the plurality of measurement items and that the determination unit determines the number of measurement items, for which measurement is to be performed, as the driving conditions.

It is preferable that the determination unit changes the driving conditions so as to increase driving power of the wearable biological sensor device so that the wearable biological sensor device can perform measurement for the plurality of measurement items in a case where the measurement period is shortened or in a case where the measured value exceeds the predicted value and that the determination unit increases the number of measurement items in a case where the determination unit determines the number of measurement items, for which measurement is to be performed, as the driving conditions.

It is preferable that the determination unit determines a measurement interval of each of the measurement items as the driving conditions.

It is preferable that the determination unit changes the driving conditions so as to increase driving power of the wearable biological sensor device in a case where the measurement period is shortened or in a case where the measured value exceeds the predicted value and that the determination unit shortens the measurement interval in a case where the determination unit determines a measurement interval of each of the measurement items as the driving conditions.

It is preferable to further comprise an instruction receiving unit that receives a manual setting instruction of the driving conditions. It is preferable that, in a case where the measurement period acquired by the second acquisition unit does not fall within a measurement allowed period estimated in a case where measurement has been performed under the driving conditions received by the instruction receiving unit, the determination unit sends a notification indicating that the measurement period exceeds the measurement allowed period.

An operation method of a biological sensor control device of the invention is an operation method of a biological sensor control device for controlling a wearable biological sensor device that is attached to a patient and that performs measurement for measurement items regarding biological information of the patient with power supplied from a built-in battery. The operation method comprises: a first acquisition step of acquiring a remaining capacity of the battery; a second acquisition step of acquiring a measurement period of the wearable biological sensor device; and a determination step of determining driving conditions of the wearable biological sensor device based on the remaining capacity and the measurement period.

Non-transitory computer readable recording medium storing an operation program of a biological sensor control device of the invention is an operation program of a biological sensor control device for controlling a wearable biological sensor device that is attached to a patient and that performs measurement for measurement items regarding biological information of the patient with power supplied from a built-in battery. The operation program causes a computer to execute: a first acquisition function of acquiring a remaining capacity of the battery; a second acquisition function of acquiring a measurement period of the wearable biological sensor device; and a determination function of determining driving conditions of the wearable biological sensor device based on the remaining capacity and the measurement period.

A biological sensor system of the invention is a biological sensor system comprising: a wearable biological sensor device that is attached to a patient and that performs measurement for measurement items regarding biological information of the patient with power supplied from a built-in battery; and a biological sensor control device that controls the wearable biological sensor device. The biological sensor control device comprises a first acquisition unit that acquires a remaining capacity of the battery, a second acquisition unit that acquires a measurement period of the wearable biological sensor device, and a determination unit that determines driving conditions of the wearable biological sensor device based on the remaining capacity and the measurement period.

According to the invention, since the measurement period and the remaining capacity of the battery of the wearable biological sensor device are acquired and the driving conditions of the wearable biological sensor device are determined based on such information, it is possible to provide a biological sensor control device, an operation method and non-transitory computer readable recording medium storing an operation program thereof, and a biological sensor system capable of effectively using the power of the battery of the wearable biological sensor device within the measurement period.

### BRIEF DESCRIPTION OF THE DRAWINGS

FIG. 1 is a diagram showing a biological sensor system.

FIG. 2 is a diagram showing various kinds of information transmitted and received between a wearable biological sensor device, a biological sensor control server, and a client terminal.

FIG. 3 is a block diagram showing a wearable biological sensor device.

FIG. 4 is a block diagram showing a computer that forms each of a biological sensor control server and a client terminal.

FIG. 5 is a block diagram showing each functional unit of a CPU of a biological sensor control server.

FIG. 6 is a diagram showing the content of a measurement value table.

FIG. 7 is a diagram showing the details of initial driving conditions.

FIG. 8 is a diagram showing the details of initial driving conditions.

FIG. 9 is a diagram showing the details of initial driving conditions.

FIG. 10 is a diagram showing the details of initial driving conditions.

FIG. 11 is a diagram showing the details of a remaining capacity and period table.

FIG. 12 is a diagram showing the details of power consumption information.

FIG. 13 is a diagram showing the details of necessity determination conditions.

FIGS. 14A to 14D show an example of changing the driving conditions so as to increase the driving power of the wearable biological sensor device in a case where the measurement period is shortened.

FIGS. 15A to 15D show an example of changing the driving conditions so as to increase the driving power of the wearable biological sensor device in a case where a measured value exceeds a predicted value.

FIGS. 16A to 16D show an example of changing the driving conditions so as to reduce the driving power of the wearable biological sensor device in a case where the measurement period is extended.

FIGS. 17A to 17D show an example of changing the driving conditions so as to reduce the driving power of the wearable biological sensor device in a case where a measured value is less than a predicted value.

FIG. 18 is a block diagram showing functional units of a CPU of a client terminal.

FIG. 19 is a diagram showing an initial setting input screen.

FIG. 20 is a diagram showing an instruction input screen.

FIG. 21 is a diagram showing a measurement value display screen.

FIG. 22 is a diagram showing a measurement period change screen.

FIG. 23 is a diagram showing a notification screen.

FIG. 24 is a flowchart showing the procedure of the biological sensor control server.

FIG. 25 is a flowchart showing the driving conditions change processing of a determination unit.

FIG. 26 is a diagram showing an example of a remaining capacity estimation graph in a case where a plurality of batteries are prepared.

FIG. 27 is a diagram showing the initial driving conditions in which a measurement allowed period is registered.

FIG. 28 is a diagram showing a second embodiment in which a notification indicating that a measurement period exceeds a measurement allowed period is sent in a case where the measurement allowed period is shorter than the measurement period.

FIG. 29 is a diagram showing a warning screen.

FIG. 30 is a diagram showing a driving conditions input region of an initial setting input screen on which a remaining capacity estimation graph and a measurement allowed period are displayed.

FIG. 31 is a diagram showing the details of driving conditions for abnormalities.

### DESCRIPTION OF THE PREFERRED EMBODIMENTS

### First Embodiment

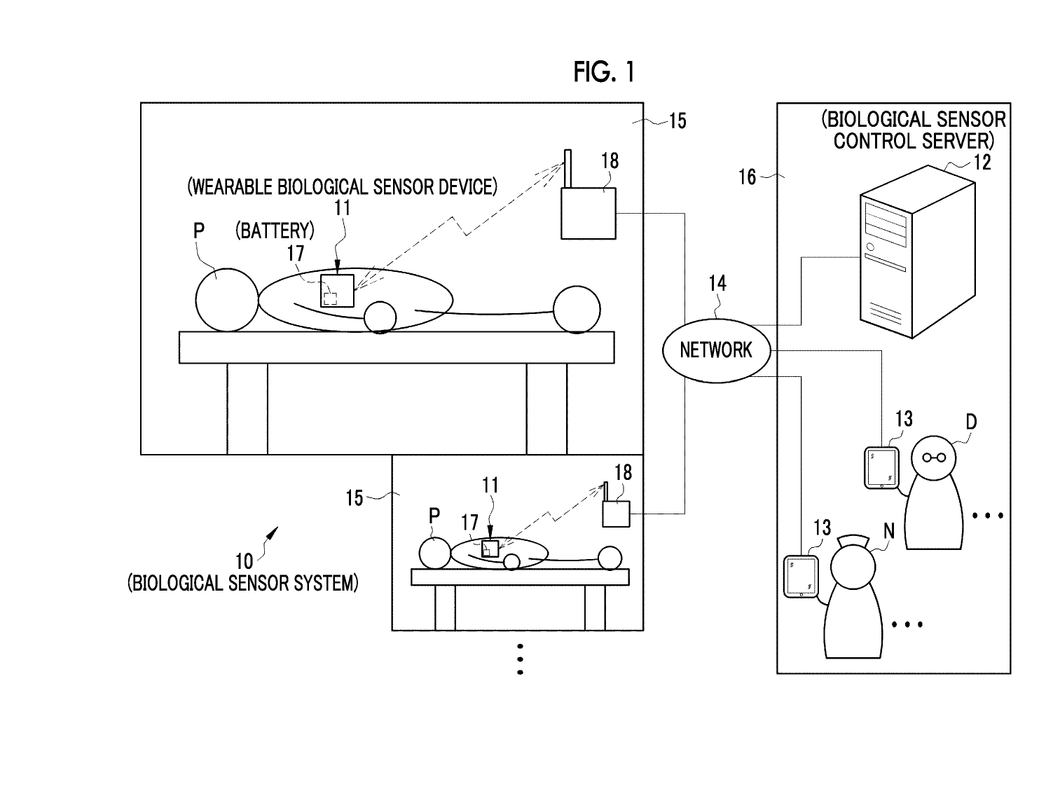

In FIG. 1, a biological sensor system 10 includes a wearable biological sensor device 11, a biological sensor control server 12 corresponding to a biological sensor control device, a client terminal 13, and the like. These are communicably connected to each other through a network 14, such as a public communication network (for example, the Internet or a wide area network (WAN). As the network 14, in consideration of the information security, a virtual private network (VPN) is constructed, or a communication protocol with a high security level, such as hypertext transfer protocol secure (HTTPS), is used.

The wearable biological sensor device 11 is attached to a patient P in order to perform measurement for various measurement items regarding the biological information of the patient P, such as a heart rate or a respiratory rate. For example, the patient P is under medical treatment at home 15, and regularly visits a medical facility 16. The patient P to whom the wearable biological sensor device 11 is attached and who needs to be monitored is selected by a doctor D. For example, a patient suffering from a disease, such as a chronic obstructive pulmonary disease (COPD) or arrhythmia or a patient who has just discharged from the hospital after surgery is selected. For the measurement period of the wearable biological sensor device 11, start date and time (for example, after the end of this examination for the patient P) and end date and time (for example, 9 o'clock in the morning of the next examination date) of the measurement are set by the doctor D.

A battery 17 is built in the wearable biological sensor device 11. The wearable biological sensor device 11 is driven by power supplied from the built-in battery 17. For example, the battery 17 is a disposable battery, such as a button battery. The wearable biological sensor device 11 has a wireless transmission and reception function so that wireless communication with a wireless transceiver 18 installed at home 15 of the patient P is possible. Accordingly, the wearable biological sensor device 11 can be used wirelessly. The wireless transceiver 18 is connected to the network 14, so that the wearable biological sensor device 11 transmits and receives information, such as measurement values of various measurement items, to and from the biological sensor control server 12 through the wireless transceiver 18.

Each of the biological sensor control server 12 and the client terminal 13 is configured by installing a control program, such as an operating system, or various application programs (hereinafter, referred to as APs) on a computer as a base, such as a server computer, a personal computer, or a workstation.

The biological sensor control server 12 is installed in the medical facility 16. The biological sensor control server 12 controls the wearable biological sensor device 11. More specifically, the biological sensor control server 12 determines the driving conditions of the wearable biological sensor device 11 based on the remaining capacity of the battery 17 and the measurement period of the wearable biological sensor device 11. In addition, the biological sensor control server 12 determines whether or not there is any abnormality in the condition of the patient P based on the measurement values.

The client terminal 13 is a tablet computer that is carried in the medical facility 16 by a medical staff member, such as a nurse N or the doctor D who monitors the condition of the patient P. The client terminal 13 is operated by the medical staff member when checking the measurement values or the like.

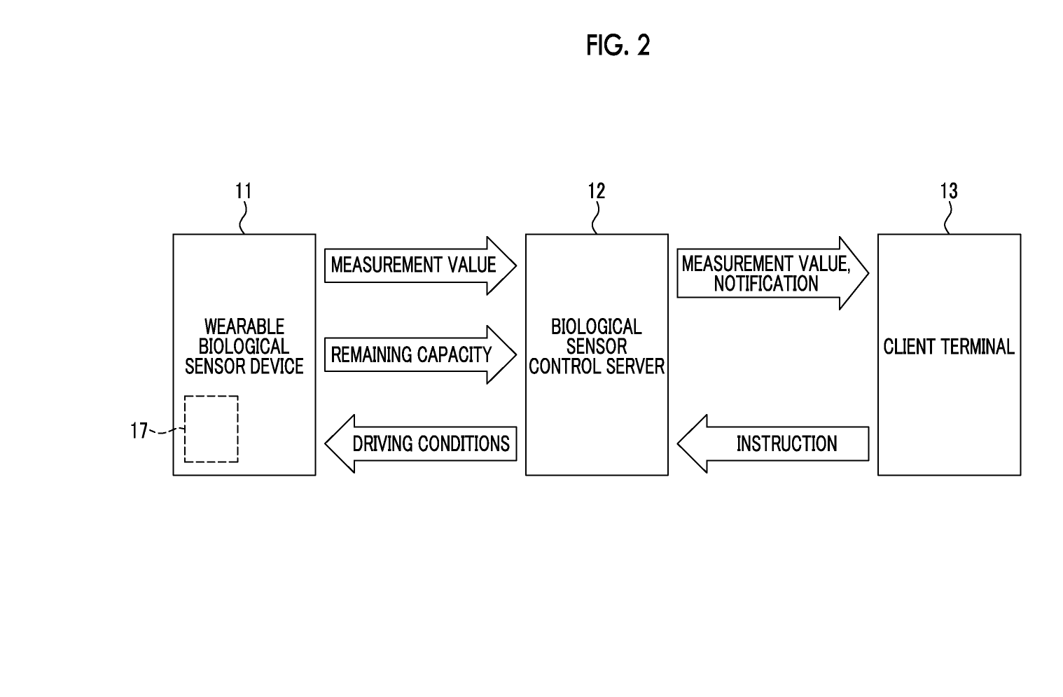

In FIG. 2, the wearable biological sensor device 11 transmits the measurement values and the remaining capacity of the battery 17 to the biological sensor control server 12. The biological sensor control server 12 transmits the driving conditions to the wearable biological sensor device 11. In addition, the biological sensor control server 12 transmits the measurement values and a notification, which indicates that it has been determined that there is a certain abnormality in the condition of the patient P, to the client terminal 13. The client terminal 13 transmits various instructions based on the hands of the medical staff member to the biological sensor control server 12.

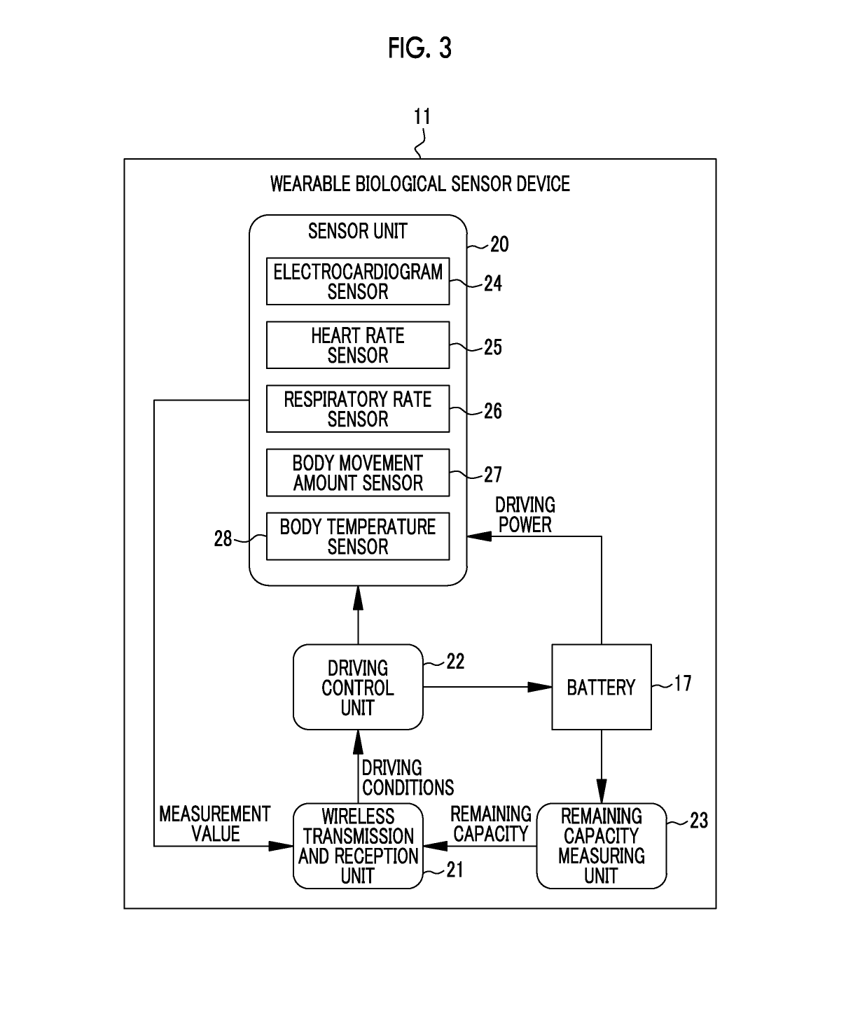

In FIG. 3, the wearable biological sensor device 11 includes a sensor unit 20, a wireless transmission and reception unit 21, a driving control unit 22, and a remaining capacity measuring unit 23 in addition to the battery 17 described above. The sensor unit 20 has a total of five sensors, that is, an electrocardiogram sensor 24, a heart rate sensor 25, a respiratory rate sensor 26, a body movement amount sensor 27, and a body temperature sensor 28. These five sensors 24 to 28 are driven by the driving power supplied from the battery 17. Each of the five sensors 24 to 28 may be a sensor that can perform measurement for a plurality of measurement items from one electrical signal (for example, measure an electrocardiogram and a heart rate from the electrical signal of the heart rate), or each of the five sensors 24 to 28 may be a sensor that can perform measurement for each measurement item from each electrical signal in a one-to-one manner.

The electrocardiogram sensor 24 measures the electrocardiographic waveform of the patient P. The heart rate sensor 25 measures the heart rate (unit: beat per minute (BPM)) of the patient P per minute, and the respiratory rate sensor 26 measures the respiratory rate (unit: breath per minute (BrPM)) of the patient P per minute. The body movement amount sensor 27 measures a body movement amount (unit: m/s²) indicating the amount of movement of the patient P. The body temperature sensor 28 measures the body temperature (unit: ° C.) of the patient P. Each of the sensors 24 to 28 outputs a measurement value, which is each measurement result, to the wireless transmission and reception unit 21.

The wireless transmission and reception unit 21 wirelessly transmits the measurement values of measurement items of the electrocardiogram, the heart rate, the respiratory rate, the body movement amount, and the body temperature from the sensors 24 to 28 to the wireless transceiver 18 together with device identification data (ID). The device ID is a symbol or a number for identifying the individual wearable biological sensor device 11, and is stored in the internal memory (not shown) of the wearable biological sensor device 11.

The wireless transmission and reception unit 21 wirelessly receives the driving conditions from the biological sensor control server 12 through the wireless transceiver 18. As the driving conditions, ON/OFF of each of the sensors 24 to 28, the measurement time of the electrocardiogram sensor 24, and the measurement interval of each of the sensors 25 to 28 other than the electrocardiogram sensor 24 are designated. The wireless transmission and reception unit 21 outputs the driving conditions to the driving control unit 22.

The driving control unit 22 controls the driving of each of the sensors 24 to 28 according to the driving conditions. Specifically, the driving control unit 22 drives a sensor, for which ON is designated in the driving conditions, by supplying the driving power from the battery 17 to the sensor. On the other hand, the driving power from the battery 17 is not supplied to a sensor, for which OFF is designated in the driving conditions. Accordingly, the sensor for which OFF is designated in the driving conditions is not driven. In the case of a sensor that can perform measurement for a plurality of measurement items from one electrical signal, ON/OFF of the operation of performing measurement for the measurement items from the electrical signal may be switched according to ON/OFF of the driving conditions instead of supplying/not supplying the driving power. Since the power is consumed according to the operation, power consumption in a case where the operation is turned off is smaller than that in a case where the operation is turned on.

The driving control unit 22 drives the electrocardiogram sensor 24 during the measurement time designated in the driving conditions. In addition, the driving control unit 22 causes the respective sensors 25 to 28 other than the electrocardiogram sensor 24 to perform measurement at the measurement intervals designated in the driving conditions.

The remaining capacity measuring unit 23 measures the remaining capacity of the battery 17 at predetermined time intervals, for example, every hour. The remaining capacity measuring unit 23 outputs the measured remaining capacity to the wireless transmission and reception unit 21. The wireless transmission and reception unit 21 wirelessly transmits the remaining capacity from the remaining capacity measuring unit 23 to the wireless transceiver 18 together with the device ID. In the present embodiment, for example, the remaining capacity is expressed as a percentage (full state: 100%, empty state: 0%). Therefore, information which indicates a percentage of the remaining capacity of the battery 17 is obtained by the remaining capacity measuring unit 23, and the information is wirelessly transmitted with the device ID. The information which indicates remaining capacity of the battery is not limited to the percentage of the remaining capacity, but may be Wh (Watt hour) etc. The following explanation will be given on the assumption that measurement is started in a state where the remaining capacity is 100%.

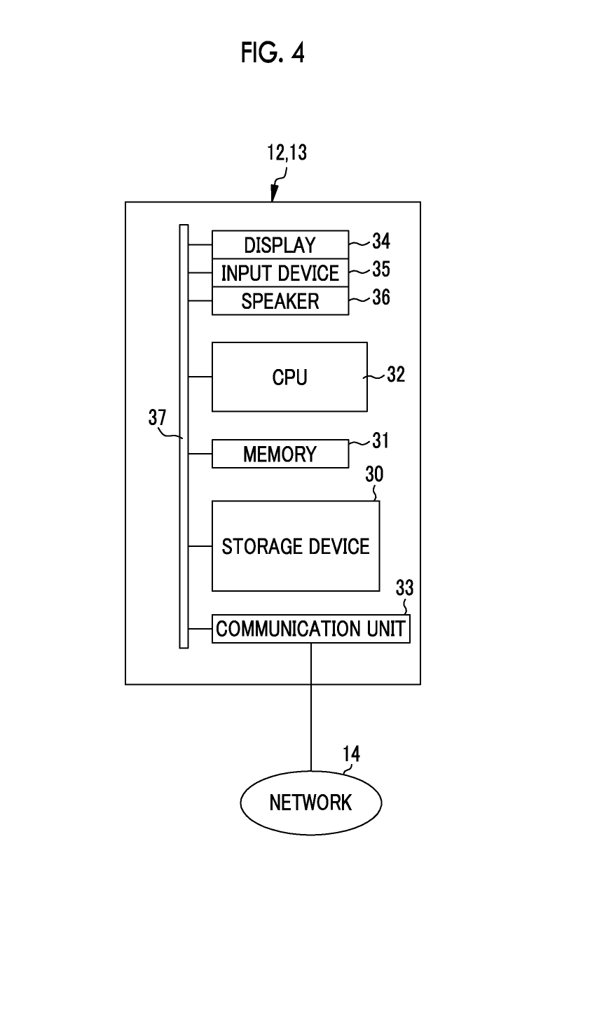

In FIG. 4, the basic configurations of computers that form the biological sensor control server 12 and the client terminal 13 are the same, and each computer includes a storage device 30, a memory 31, a central processing unit (CPU) 32, a communication unit 33, a display 34, an input device 35, and a speaker 36. These are connected to each other through a data bus 37.

The storage device 30 is a hard disk drive, which is built into a computer that forms the biological sensor control server 12 or the like or which is connected to the computer through a cable or a network, or a disk array formed by connecting a plurality of hard disk drives. A control program such as an operating system, various APs, and various kinds of data associated with these programs are stored in the storage device 30.

The memory 31 is a work memory required when the CPU 32 executes processing. The CPU 32 performs overall control of each unit of the computer by loading a program stored in the storage device 30 to the memory 31 and executing the processing according to the program.

The communication unit 33 is a network interface to perform transmission control of various kinds of information through the network 14. The display 34 displays various screens corresponding to the operation of the input device 35, such as a mouse, a keyboard, or a touch panel. The screen has an operation function based on the graphical user interface (GUI). Each computer that forms the biological sensor control server 12 or the like receives an input of an operation instruction from the input device 35 through the screen.

In the following explanation, for the sake of distinction, a suffix “A” is attached to the reference numeral of each unit of the computer that forms the biological sensor control server 12, and a suffix “B” is attached to the reference numeral of each unit of the computer that forms the client terminal 13.

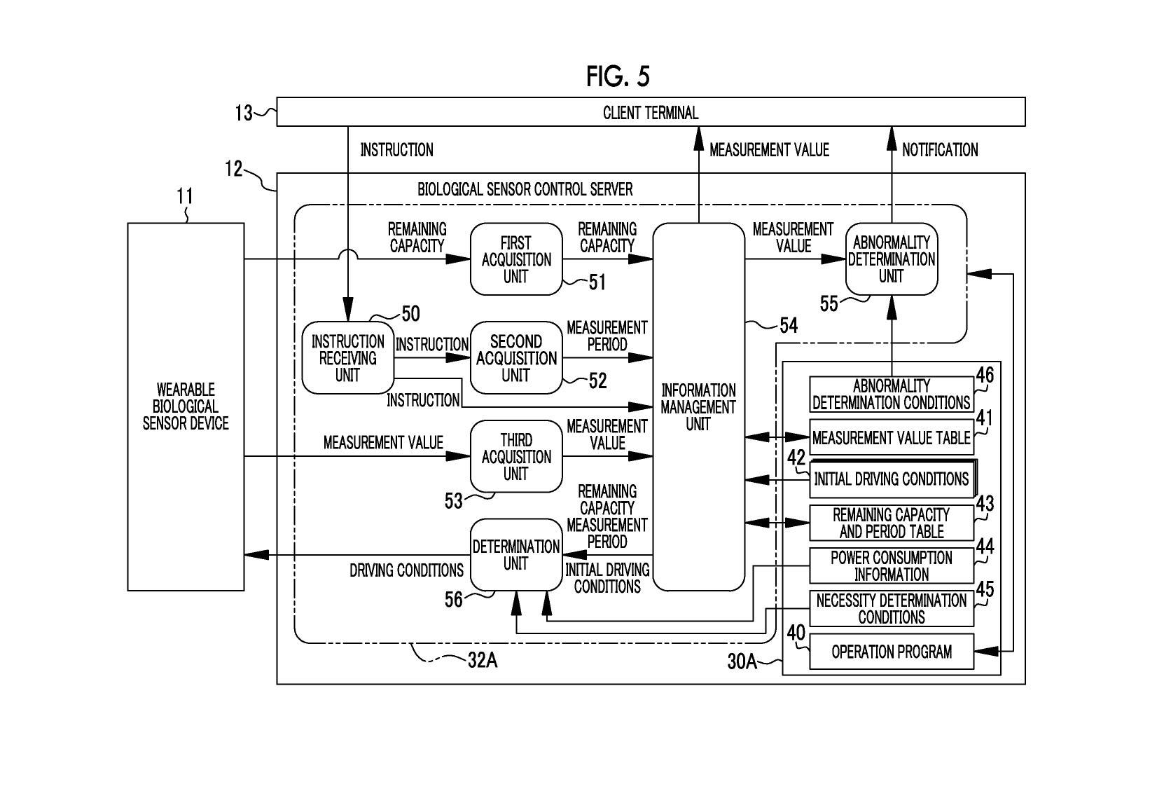

In FIG. 5, an operation program 40 as an AP is stored in the storage device 30A of the biological sensor control server 12. The operation program 40 is an AP for making the computer that forms the biological sensor control server 12 function as a biological sensor control device.

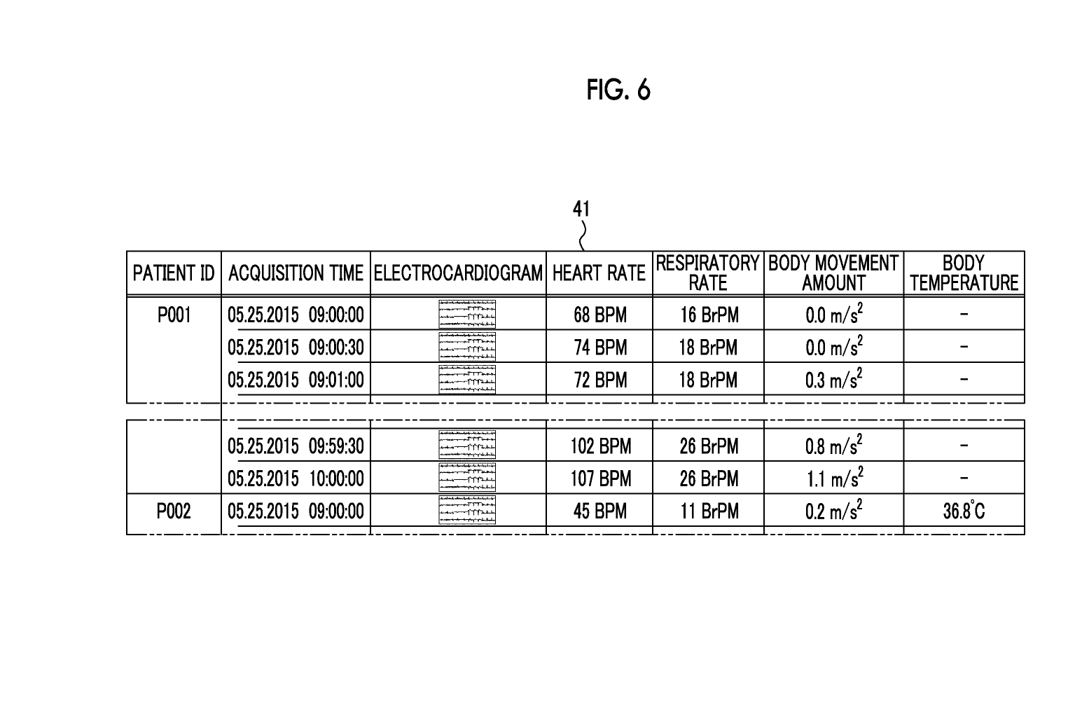

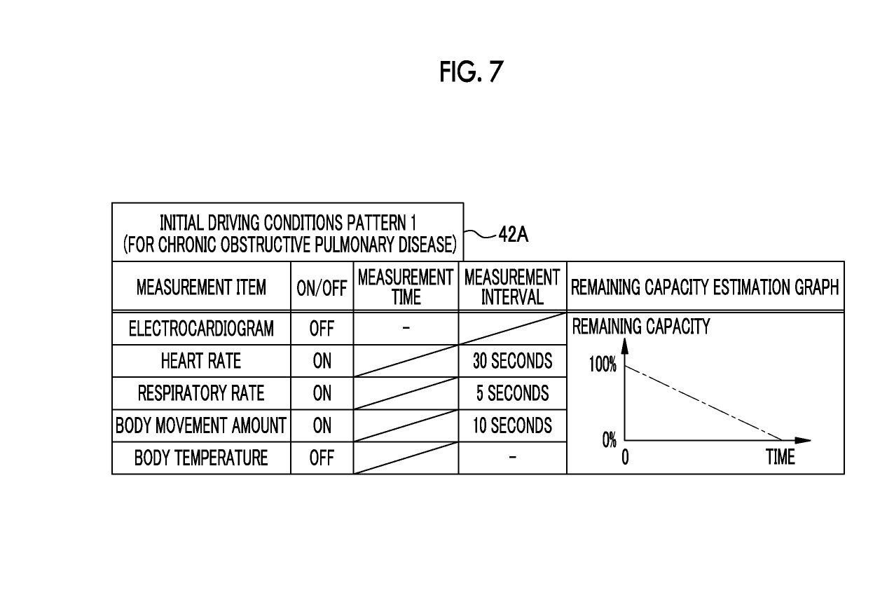

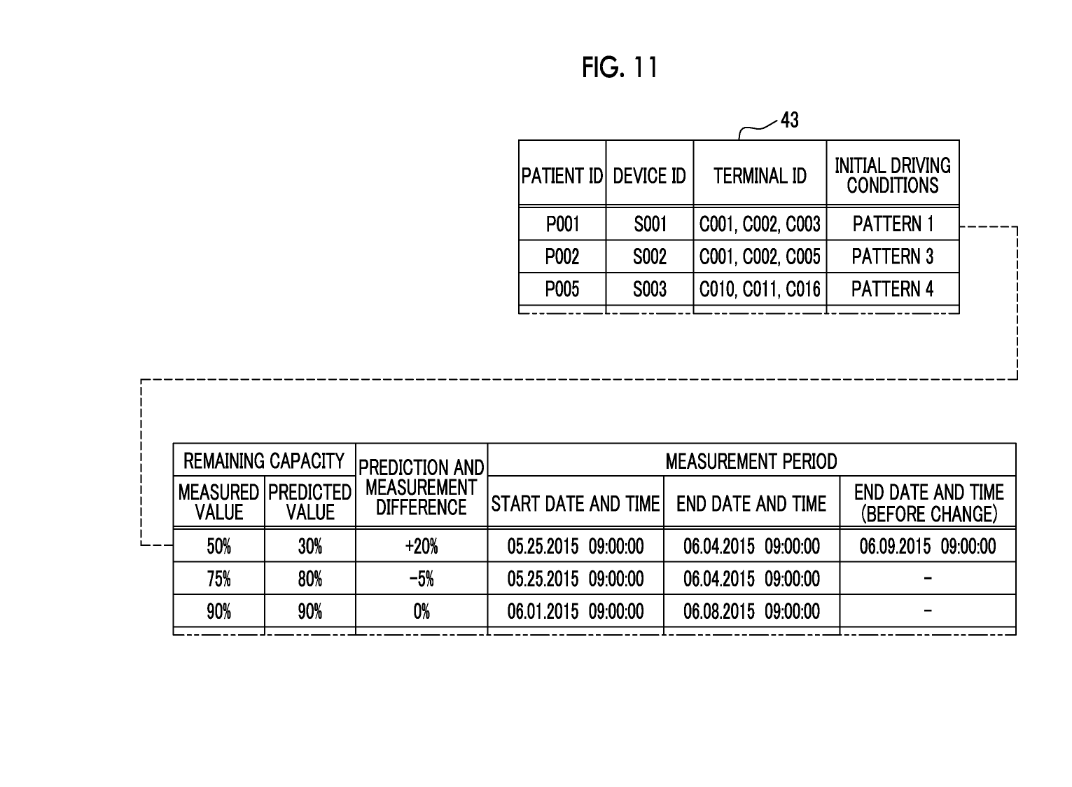

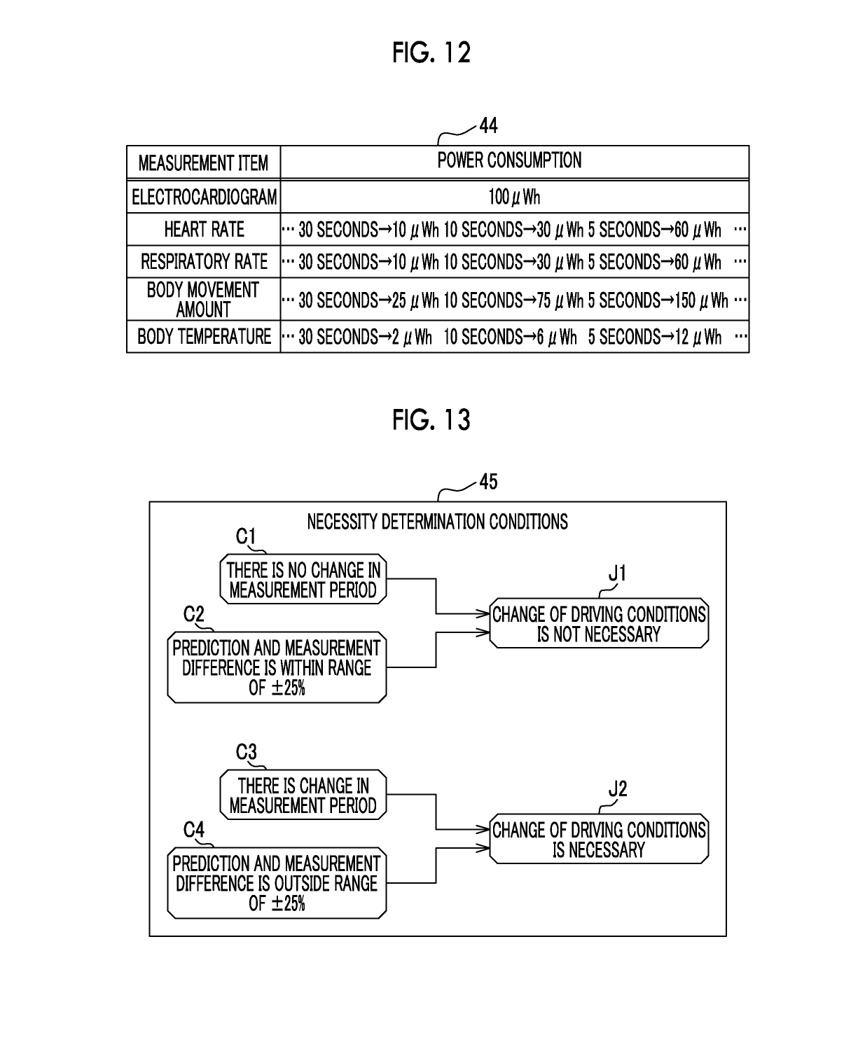

Not only the operation program 40 but also a measurement value table 41 (refer to FIG. 6), a plurality of kinds of initial driving conditions 42 (refer to FIGS. 7 to 10), a remaining capacity and period table 43 (refer to FIG. 11), power consumption information 44 (refer to FIG. 12), necessity determination conditions 45 (refer to FIG. 13), and abnormality determination conditions 46 are stored in the storage device 30A.

When the operation program 40 is started, the CPU 32A of the biological sensor control server 12 cooperates with the memory 31 or the like to function as an instruction receiving unit 50, a first acquisition unit 51, a second acquisition unit 52, a third acquisition unit 53, an information management unit 54, an abnormality determination unit 55, and a determination unit 56.

The instruction receiving unit 50 receives various instructions from the client terminal 13. Various instructions include an initial setting instruction, a measurement period change instruction, a measurement value request instruction, and the like. The initial setting instruction is an instruction given before starting the measurement for the measurement items using the wearable biological sensor device 11. A patient ID that is a symbol or a number for identifying the patient P to whom the wearable biological sensor device 11 is attached, a device ID of the wearable biological sensor device 11, a terminal ID that is a symbol or a number for identifying the client terminal 13 carried by a medical staff member in charge of the patient P, a measurement period (start date and time and end date and time) of the wearable biological sensor device 11, and the initial driving conditions 42 are included in the initial setting instruction. Since the initial driving conditions 42 are included in the initial setting instruction, the initial setting instruction corresponds to a manual setting instruction of the driving conditions.

The measurement period change instruction is an instruction to change the end date and time of the measurement period during the measurement period set in the initial setting instruction. The measurement value request instruction is an instruction to request the display of the latest measurement value at that point in time. A patient ID is included in the measurement period change instruction and the measurement value request instruction. In addition, a terminal ID is included in the measurement value request instruction. The instruction receiving unit 50 outputs the initial setting instruction and the measurement period change instruction to the second acquisition unit 52, and outputs the initial setting instruction and the measurement value request instruction to the information management unit 54.

The first acquisition unit 51 acquires the remaining capacity from the wearable biological sensor device 11. The second acquisition unit 52 acquires the measurement period included in the initial setting instruction from the instruction receiving unit 50 and the end date and time after s change, which is included in the measurement period change instruction, as a measurement period of the wearable biological sensor device 11. The third acquisition unit 53 acquires the measurement value from the wearable biological sensor device 11. These acquisition units 51 to 53 output the remaining capacity, the measurement period, and the measurement value, which have been acquired, to the information management unit 54, respectively.

The information management unit 54 registers, in the remaining capacity and period table 43, the remaining capacity from the first acquisition unit 51, the measurement period from the second acquisition unit 52, and information other than the measurement period, such as a patient ID or a device ID, which is included in the initial setting instruction from the instruction receiving unit 50. In addition, the information management unit 54 registers the measurement value from the third acquisition unit 53 in the measurement value table 41.

The information management unit 54 reads out the initial driving conditions 42, which are selected in the initial setting instruction, from a plurality of kinds of initial driving conditions 42 stored in the storage device 30A, and transmits the read-out initial driving conditions 42 to the determination unit 56 together with the measurement period and the device ID. The information management unit 54 transmits information regarding the remaining capacity of the remaining capacity and period table 43 and information regarding the measurement period to the determination unit 56. In addition, the information management unit 54 transmits the measurement value of the measurement value table 41 to the abnormality determination unit 55.

In response to the measurement value request instruction from the instruction receiving unit 50, the information management unit 54 reads out the latest measurement value of the patient P corresponding to the patient ID included in the measurement value request instruction from the measurement value table 41, and transmits the read-out measurement value to the client terminal 13 that is the transmission source of the measurement value request instruction. The client terminal 13 that is the transmission source of the measurement value request instruction can be specified by the terminal ID included in the measurement value request instruction.

The abnormality determination unit 55 determines whether or not a measurement item satisfies the abnormality determination conditions 46 based on the measurement value from the information management unit 54. In a case where a measurement item satisfies the abnormality determination conditions 46, the abnormality determination unit 55 transmits a notification, which indicates that it has been determined that a measurement item satisfies the abnormality determination conditions 46, to the client terminal 13.

The abnormality determination conditions 46 are conditions for determining whether or not any abnormality in the condition of the patient P has occurred based on each measurement item, such as a heart rate and a respiratory rate. A threshold value of the measurement value for each measurement item, for example, a heart rate of 100 BPM or a respiratory rate of 25 BrPM, and a period, for example, 1 minute, are set in the abnormality determination conditions 46. The abnormality determination unit 55 determines that the measurement item satisfies the abnormality determination conditions 46 in a case where a state where the measurement value is equal to or greater than the threshold value continues for a set period or more.

The determination unit 56 transmits the initial driving conditions 42 from the information management unit 54 to the wearable biological sensor device 11 together with the measurement period. In addition, the determination unit 56 determines the driving conditions of the wearable biological sensor device 11 based on the information regarding the remaining capacity and the information regarding the measurement period from the information management unit 54. The determination unit 56 transmits the determined driving conditions to the wearable biological sensor device 11 together with the measurement period.

As shown in FIG. 6, in the measurement value table 41, each measurement value and the acquisition time, at which each measurement value such as a heart rate and a respiratory rate has been acquired by the third acquisition unit 53, are registered for each patient ID. All of the measurement values acquired during the measurement period of one patient P are registered in the measurement value table 41. FIG. 6 shows an example of the driving conditions of the biological sensor device 11 of the patient P of, for example, patient ID: P001 in which ON is designated for the sensors 24 to 27 other than the body temperature sensor 28, “always” is designated as the measurement time of the electrocardiogram sensor 24, and 30 seconds is designated as the measurement interval of the sensors 25 to 27 other than the electrocardiogram sensor 24. In this case, in the field of the body temperature of patient ID: P001, OFF is designated as driving conditions. Accordingly, needless to say, no measurement value is registered in the field of the body temperature of patient ID: P001.

When transmitting the measurement value from the measurement value table 41 to the abnormality determination unit 55, the information management unit 54 attaches the patient ID. In addition, the abnormality determination unit 55 attaches the patient ID to the notification indicating that it has been determined that the measurement item satisfies the abnormality determination conditions 46.

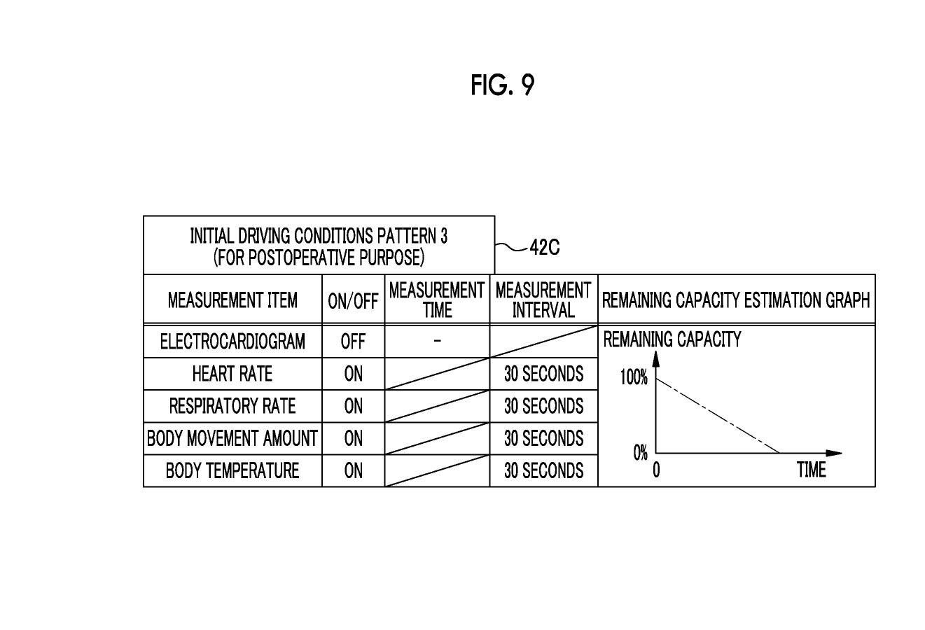

Initial driving conditions 42A to 42D of patterns 1 to 4 shown in FIGS. 7 to 10 are examples of a plurality of kinds of initial driving conditions 42. As the initial driving conditions 42A to 42D, as described above, ON/OFF of each of the sensors 24 to 28 for measuring each measurement item, such as a heart rate and a respiratory rate, the measurement time of the electrocardiogram sensor 24, and the measurement interval of each of the sensors 25 to 28 other than the electrocardiogram sensor 24 are registered. In a case where OFF is designated for the electrocardiogram sensor 24, nothing is registered in the field of the measurement time (refer to FIGS. 7 and 9). In addition, in a case where OFF is designated for each of the sensors 25 to 28 other than the electrocardiogram sensor 24, nothing is registered in the field of the measurement interval of the sensor (refer to the field of the body temperature in FIG. 7).

Data indicating a remaining capacity estimation graph, in which the vertical axis indicates a remaining capacity and the horizontal axis indicates time and in which the remaining capacity with time until the remaining capacity becomes 0% from 100% is expressed with a line segment shown by a one-dot chain line, is registered in the initial driving conditions 42A to 42D. From the remaining capacity estimation graph, it is possible to calculate a predicted value of the remaining capacity at a certain point in time of the measurement period.

The initial driving conditions 42A of the pattern 1 shown in FIG. 7 are for patients with a chronic obstructive pulmonary disease, and ON is designated for the heart rate sensor 25, the respiratory rate sensor 26, and the body movement amount sensor 27. As the measurement interval, 30 seconds is designated for the heart rate, while 5 seconds is designated for the respiratory rate and 10 seconds is designated for the body movement amount since it is necessary to focus on monitoring the respiratory rate and the body movement amount in the chronic obstructive pulmonary disease.

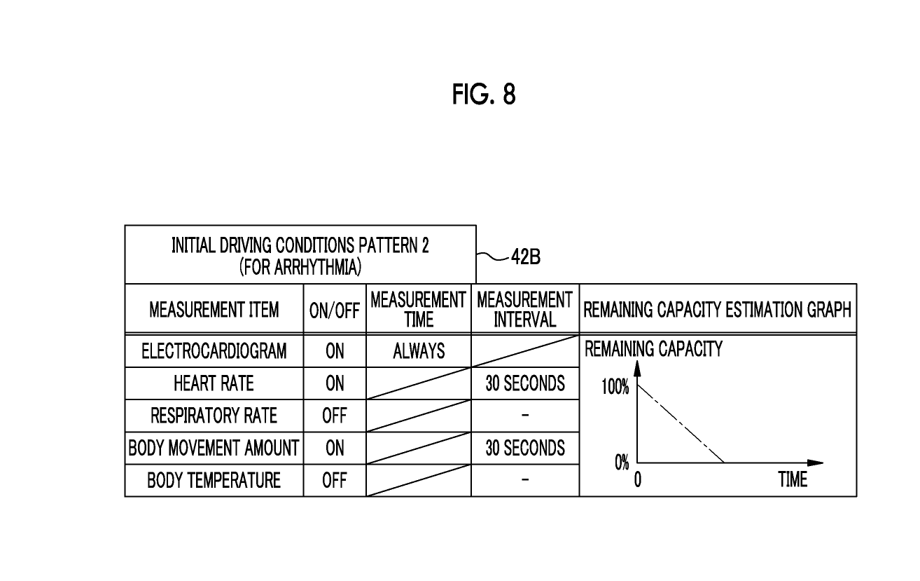

The initial driving conditions 42B of the pattern 2 shown in FIG. 8 are for patients with arrhythmia, and ON is designated for the electrocardiogram sensor 24, the heart rate sensor 25, and the body movement amount sensor 27. In the case of arrhythmia, it is necessary to focus on monitoring the electrocardiogram. Accordingly, “always” is designated as the measurement time of the electrocardiogram sensor 24.

The initial driving conditions 42C of the pattern 3 shown in FIG. 9 are for postoperative patients, and ON is designated for the sensors 25 to 28 other than the electrocardiogram sensor 24 and 30 seconds is uniformly designated as the measurement interval.

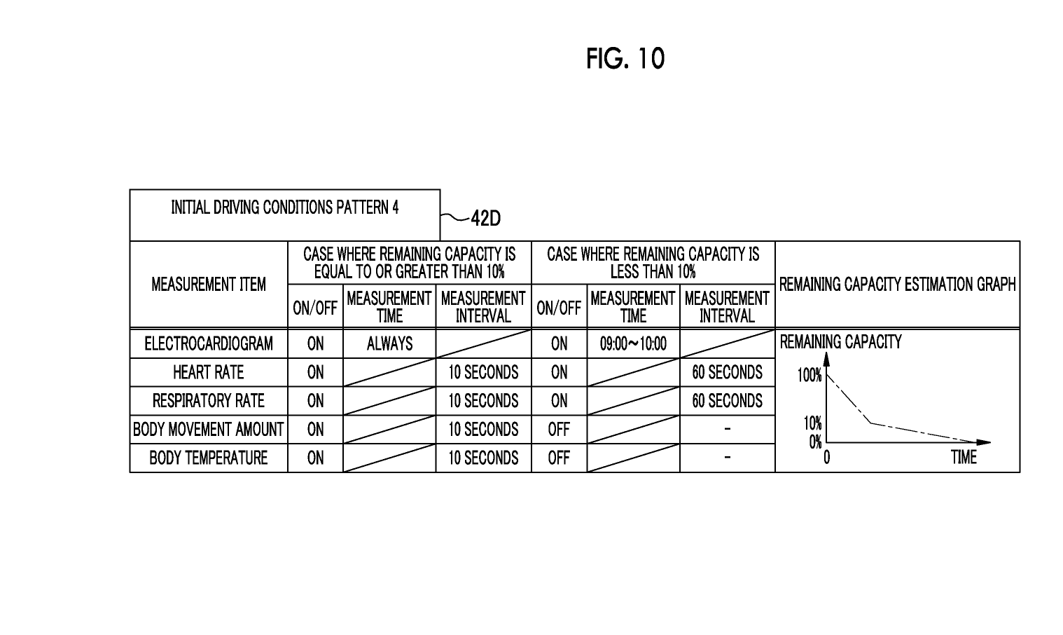

In the initial driving conditions 42D of the pattern 4 shown in FIG. 10, designation in a case where the remaining capacity is 10% or more and designation in a case where the remaining capacity is less than 10% are different. That is, in a case where the remaining capacity is 10% or more, ON is designated for all of the sensors 24 to 28, “always” is designated as the measurement time of the electrocardiogram sensor 24, and 10 seconds is designated as the measurement interval of each of the sensors 25 to 28 other than the electrocardiogram sensor 24. On the other hand, in a case where the remaining capacity is less than 10%, ON is designated for the electrocardiogram sensor 24, the heart rate sensor 25, and the respiratory rate sensor 26, 09:00 to 10:00 is designated as the measurement time of the electrocardiogram sensor 24, and 60 seconds is designated as the measurement interval of the heart rate sensor 25 and the respiratory rate sensor 26.

Thus, in the initial driving conditions 42D, in a case where the remaining capacity is 10% or more, all of the sensors 24 to 28 are turned on so that the electrocardiogram is measured always and each of the sensors 25 to 28 performs measurement at measurement intervals of 10 seconds, which is relatively short. In addition, in a case where the remaining capacity is less than 10%, the body movement amount sensor 27 and the body temperature sensor 28 are turned off so that the electrocardiogram is measured in the limited time and the heart rate sensor 25 and the respiratory rate sensor 26 perform measurement at measurement intervals of 60 seconds, which is relatively long. Accordingly, the initial driving conditions 42D can be said to be a type in which the first half of the measurement period is emphasized.

In a remaining capacity estimation graph in the case of the initial driving conditions 42D shown in FIG. 10, unlike remaining capacity estimation graphs of the initial driving conditions 42A to 42C shown in FIGS. 7 to 9 in which the remaining capacity decreases in a straight line from 100% to 0% with time, a decrease in the remaining capacity from 100% to 10% is relatively steep and a decrease in the remaining capacity from 10% to 0% is relatively gentle. That is, the way of decrease in the remaining capacity is divided into two stages. In addition, as the initial driving conditions 42, the initial driving conditions 42 of various patterns may be prepared without being limited to the initial driving conditions 42A to 42D exemplified in FIGS. 7 to 10.

As shown in FIG. 11, in the remaining capacity and period table 43, a device ID of the wearable biological sensor device 11 attached to each patient P, a terminal ID of the client terminal 13 held by a medical staff member in charge of each patient P, and a pattern of the initial driving conditions 42 selected in the initial setting instruction are registered for each patient ID.

In addition, a remaining capacity, a prediction and measurement difference, and a measurement period are registered in the remaining capacity and period table 43 for each patient ID. The field of the remaining capacity is divided into two subfields of a measured value and a predicted value. The remaining capacity acquired by the first acquisition unit 51, that is, a remaining capacity measured by the remaining capacity measuring unit 23 of the wearable biological sensor device 11, is registered in the subfield of the measured value. A predicted value of the remaining capacity at the time of acquiring the remaining capacity in the first acquisition unit 51 is registered in the subfield of the predicted value. A predicted value can be calculated from the remaining capacity estimation graph of the initial driving conditions 42 of the pattern registered in the field of initial driving conditions.

The prediction and measurement difference is a difference between the predicted value and the measured value. For example, in the case of patient ID: P001, the measured value is 50% and the predicted value is 30%. Accordingly, the prediction and measurement difference is 50−30=+20%. As the cause of the prediction and measurement difference, the use environment of the wearable biological sensor device 11, such as the room temperature in the home 15 of the patient P or a communication state between the wireless transceiver 18 and the wireless transmission and reception unit 21, is different from the assumption when creating the remaining capacity estimation graph.

In the field of the measurement period, subfields of start date and time and end date and time and a subfield of end date and time before being changed in a measurement period change instruction (end date and time (before change)) are prepared. In a case where there is no measurement period change instruction, start date and time and end date and time set in the initial setting instruction are registered in the subfields of start date and time and end date and time, and nothing is registered in the subfield of end date and time (before change). In FIG. 11, patient IDs of P002 and P005 correspond to this.

On the other hand, in a case where there is a measurement period change instruction, there is no change in the fact that the start date and time set in the initial setting instruction is registered in the subfield of start date and time, but end date and time after change included in the measurement period change instruction is registered in the subfield of end date and time. In addition, end date and time registered in the subfield of end date and time before the measurement period change instruction is rewritten in the subfield of end date and time (before change). In FIG. 11, the patient ID: P001 corresponds to this, for which the original end date and time is 09:00:00 of 2015.06.09 but the end date and time has been changed to 09:00:00 of 2015.06.04 by the measurement period change instruction, for example, by advancing the next examination date depending on the circumstances of the patient P.

The information management unit 54 specifies a patient ID, for which a measurement value is to be registered in the measurement value table 41, with reference to the remaining capacity and period table 43. Specifically, the information management unit 54 recognizes a device ID and a corresponding patient ID, which are transmitted together with a measurement value from the wearable biological sensor device 11, in the remaining capacity and period table 43, and sets the recognized patient ID as a measurement value registration destination.

The abnormality determination unit 55 specifies the client terminal 13, to which a notification is to be transmitted, with reference to the remaining capacity and period table 43. Specifically, the abnormality determination unit 55 recognizes a patient ID and a corresponding terminal ID, which are included in the measurement value from the information management unit 54, in the remaining capacity and period table 43, and sets the client terminal 13 of the recognized terminal ID as a notification transmission destination.

In FIG. 12, the power consumption of each of the sensors 24 to 28 is registered in the power consumption information 44. For each of the sensors 25 to 28 other than the electrocardiogram sensor 24, power consumption is registered every measurement interval. The remaining capacity estimation graph is created based on the power consumption information 44. By referring to the power consumption information 44, the determination unit 56 can determine the driving conditions in which the remaining capacity becomes a preset value or less at the end of the measurement period.

The information management unit 54 outputs a measured value and a prediction and measurement difference to the determination unit 56 as information regarding the remaining capacity. In a case where there is a measurement period change instruction, the information management unit 54 outputs end date and time before change and end date and time after change, as information regarding the measurement period, to the determination unit 56. When outputting such information to the determination unit 56, the information management unit 54 attaches a device ID to the information.

The determination unit 56 specifies the wearable biological sensor device 11, to which the initial driving conditions 42 or the driving conditions after change are to be transmitted, based on the device ID transmitted from the information management unit 54 together with the initial driving conditions 42, the prediction and measurement difference, and the like.

The determination unit 56 determines whether or not to change the driving conditions during the measurement period. More specifically, the determination unit 56 monitors a change in the measurement period and a prediction and measurement difference during the measurement period, and determines whether or not to change the driving conditions based on the monitoring results.

In FIG. 13, the necessity determination conditions 45 are conditions used when the determination unit 56 determines whether or not to change the driving conditions during the measurement period. In a case where there is no change in a measurement period since there is no measurement period change instruction (C1), the determination unit 56 determines that the change of the driving conditions is not necessary (J1). In a case where the prediction and measurement difference is within a range of ±25% that is a predetermined range (C2), the determination unit 56 determines that the change of the driving conditions is not necessary similarly (J1).

On the other hand, in a case where the end date and time before change and the end date and time after change are received from the information management unit 54, the determination unit 56 recognizes that the measurement period has been changed (C3), and determines that the change of the driving conditions is necessary (J2). In a case where the prediction and measurement difference is outside the range of ±25% (C4), the determination unit 56 determines that the change of the driving conditions is necessary (J2). In this case, the determination unit 56 changes the driving conditions.

In a case where the measurement period is shortened or in a case where the measured value exceeds the predicted value, the determination unit 56 changes the driving conditions so as to increase the driving power of the wearable biological sensor device 11. More specifically, in a case where the measurement period is shortened or in a case where the measured value exceeds the predicted value, the determination unit 56 increases the number of measurement items and/or shortens the measurement interval.

On the other hand, in a case where the measurement period is extended or in a case where the measured value is less than the predicted value, the determination unit 56 changes the driving conditions so as to reduce the driving power of the wearable biological sensor device 11. More specifically, in a case where the measurement period is extended or in a case where the measured value is less than the predicted value, the determination unit 56 reduces the number of measurement items and/or increases the measurement interval.

By referring to the power consumption information 44, the determination unit 56 determines the driving conditions in which the remaining capacity becomes a preset value or less at the end of the measurement period.

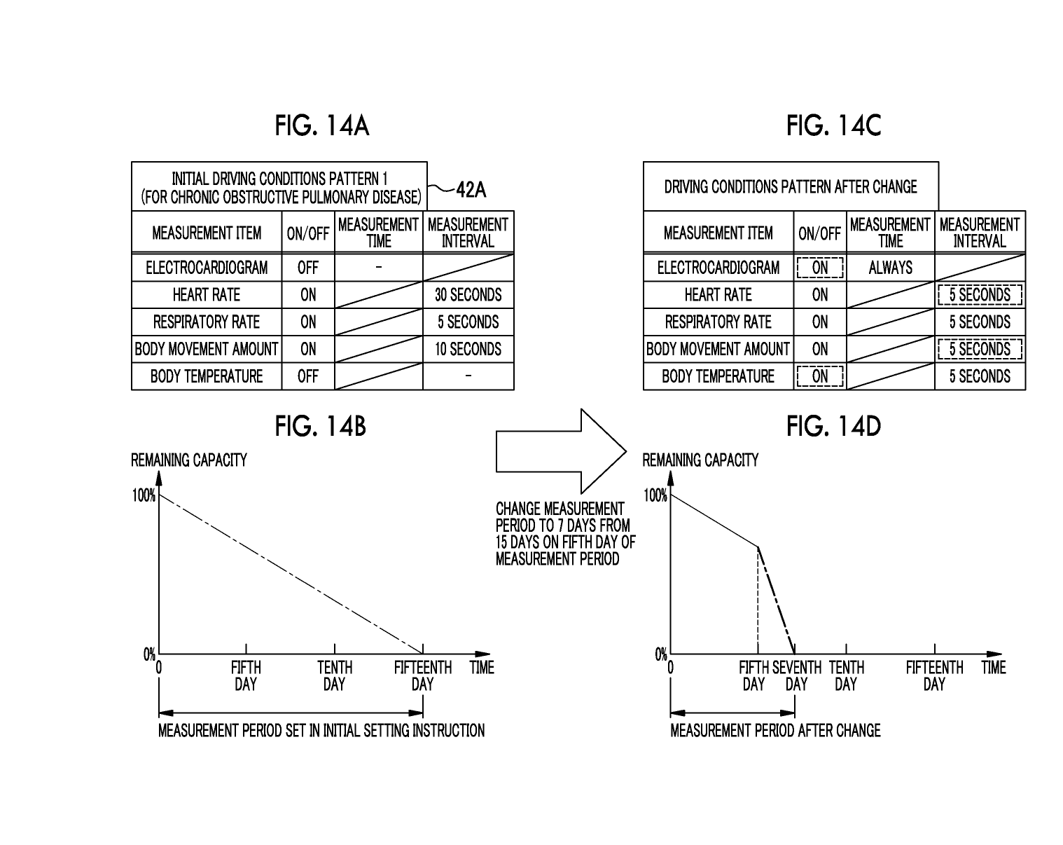

FIGS. 14A to 14D show an example of changing the driving conditions so as to increase the driving power of the wearable biological sensor device 11 in a case where the measurement period is shortened. FIGS. 14A and 14B on the left side of the arrow show a state before the instruction receiving unit 50 receives a measurement period change instruction, and FIGS. 14C and 14D on the right side of the arrow show a state after changing the driving conditions in response to a measurement period change instruction received by the instruction receiving unit 50.

Before the instruction receiving unit 50 receives a measurement period change instruction, the initial driving conditions 42A of the pattern 1 shown in FIG. 7 are selected as shown in FIG. 14A, and 15 days are set as a measurement period in the initial setting instruction as shown in FIG. 14B. In this state, as shown by a one-dot chain line in FIG. 14B, the predicted value is estimated to become 0% of the set value exactly on the fifteenth day that is the end date and time of the measurement period.

For example, in a case where there is a measurement period change instruction on the fifth day of the measurement period to change the measurement period from fifteen days to seven days, that is, shorten the measurement period, the determination unit 56 increases the number of measurement items by changing the designation of ON/OFF of the electrocardiogram sensor 24 and the body temperature sensor 28 to ON from OFF, as shown in FIG. 14C. In addition, the measurement interval is shortened by changing the measurement interval of the heart rate sensor 25 from 30 seconds to 5 seconds and changing the measurement interval of the body movement amount sensor 27 to 5 seconds from 10 seconds. In this case, as shown in FIG. 14D, the determination unit 56 adjusts the measurement interval and the number of measurement items to be increased, based on the measured value shown by the solid line and the power consumption information 44, so that the predicted value after the fifth day shown by the one-dot chain line becomes 0% of the set value exactly on the seventh day that is the end date and time after change.

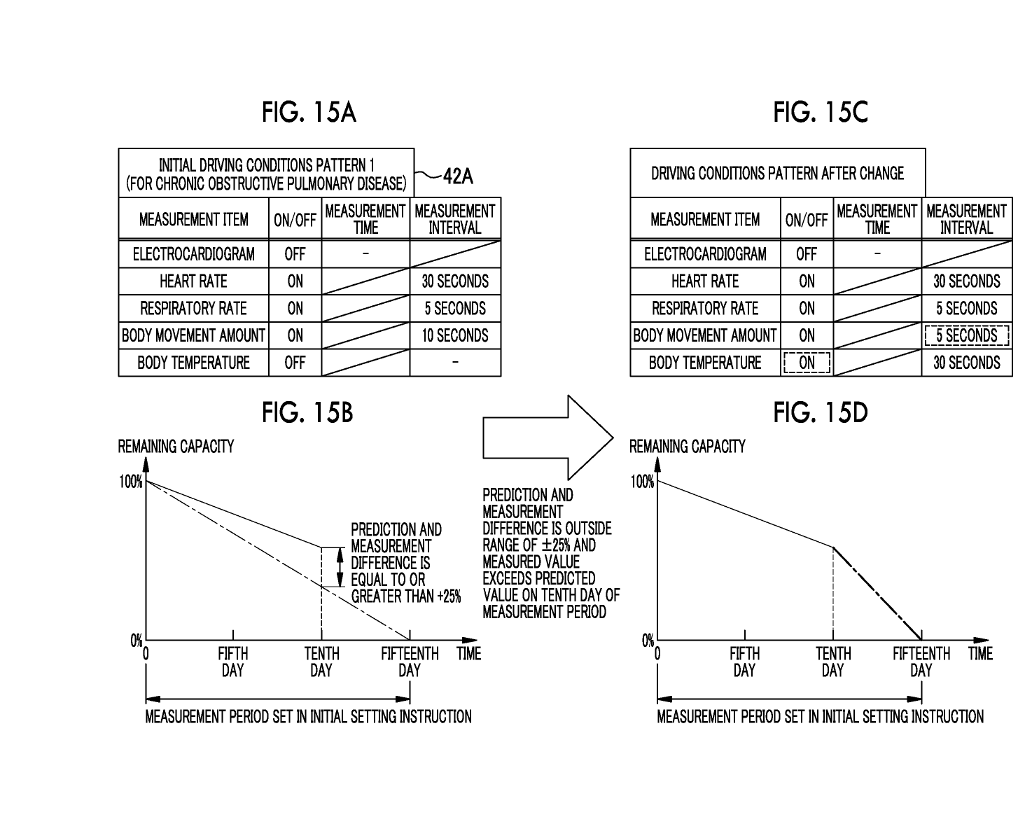

FIGS. 15A to 15D show an example of changing the driving conditions so as to increase the driving power of the wearable biological sensor device 11 in a case where the measured value of the remaining capacity of the battery 17 exceeds the predicted value. FIGS. 15A and 15B on the left side of the arrow show a state before changing the driving conditions, and FIGS. 15C and 15D on the right side of the arrow show a state after changing the driving conditions. Before changing the driving conditions, as shown in FIGS. 15A and 15B, the initial driving conditions 42A of the pattern 1 shown in FIG. 7 are selected and 15 days are set as a measurement period in the initial setting instruction, as in FIGS. 14A and 14B.

As shown in FIG. 15B, for example, in a case where the prediction and measurement difference between the measured value shown by the solid line and the predicted value shown by the one-dot chain line is +25% or more on the tenth day of the measurement period, that is, the prediction and measurement difference is outside the range of ±25% and the measured value exceeds the predicted value, the determination unit 56 increases the number of measurement items by changing the designation of ON/OFF of the body temperature sensor 28 to ON from OFF, as shown in FIG. 15C. In addition, the measurement interval is shortened by changing the measurement interval of the body movement amount sensor 27 to 5 seconds from 10 seconds. In this case, as shown in FIG. 15D, the determination unit 56 adjusts the measurement interval and the number of measurement items to be increased, based on the measured value shown by the solid line and the power consumption information 44, so that the predicted value after the tenth day shown by the one-dot chain line becomes 0% of the set value exactly on the fifteenth day that is the end date and time.

In the cases shown in FIGS. 14A to 14D and 15A to 15D, the driving conditions are changed so as to increase the driving power of the wearable biological sensor device 11. Accordingly, in a remaining capacity estimation graph after the change of the driving conditions, the slope is steep compared with that before the change of the driving conditions.

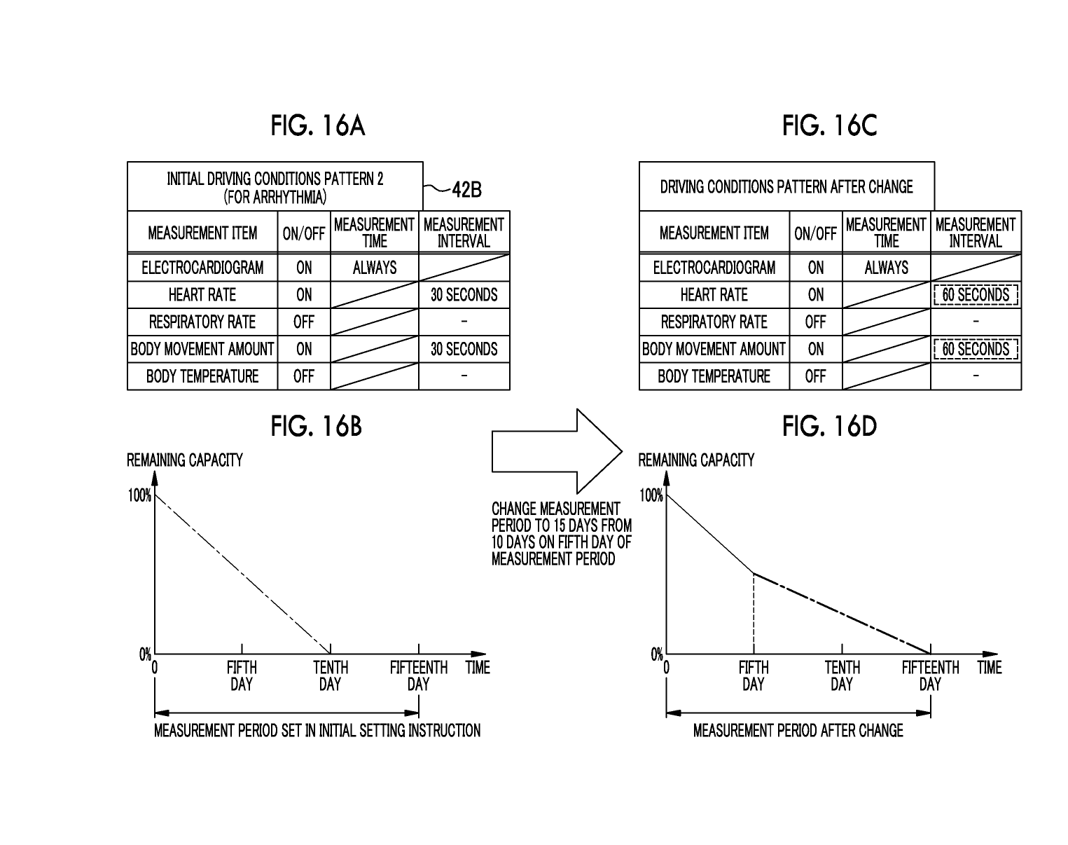

FIGS. 16A to 16D show an example of changing the driving conditions so as to reduce the driving power of the wearable biological sensor device 11 in a case where the measurement period is extended. FIGS. 16A and 16B on the left side of the arrow show a state before the instruction receiving unit 50 receives a measurement period change instruction, and FIGS. 16C and 16D on the right side of the arrow show a state after changing the driving conditions in response to a measurement period change instruction received by the instruction receiving unit 50.

Before the instruction receiving unit 50 receives a measurement period change instruction, the initial driving conditions 42B of the pattern 2 shown in FIG. 8 are selected as shown in FIG. 16A, and 10 days are set as a measurement period in the initial setting instruction as shown in FIG. 16B. In this state, as shown by a one-dot chain line in FIG. 16B, the predicted value is estimated to become 0% of the set value exactly on the tenth day that is the end date and time of the measurement period.

For example, in a case where there is a measurement period change instruction on the fifth day of the measurement period to change the measurement period from ten days to fifteen days, that is, increase the measurement period, the determination unit 56 increases the measurement period by changing the measurement periods of the electrocardiogram sensor 25 and the body movement amount sensors 27 to 60 seconds from 30 seconds, as shown in FIG. 16C. In this case, as shown in FIG. 16D, the determination unit 56 adjusts the measurement interval, based on the measured value shown by the solid line and the power consumption information 44, so that the predicted value after the fifth day shown by the one-dot chain line becomes 0% of the set value exactly on the fifteenth day that is the end date and time after change.

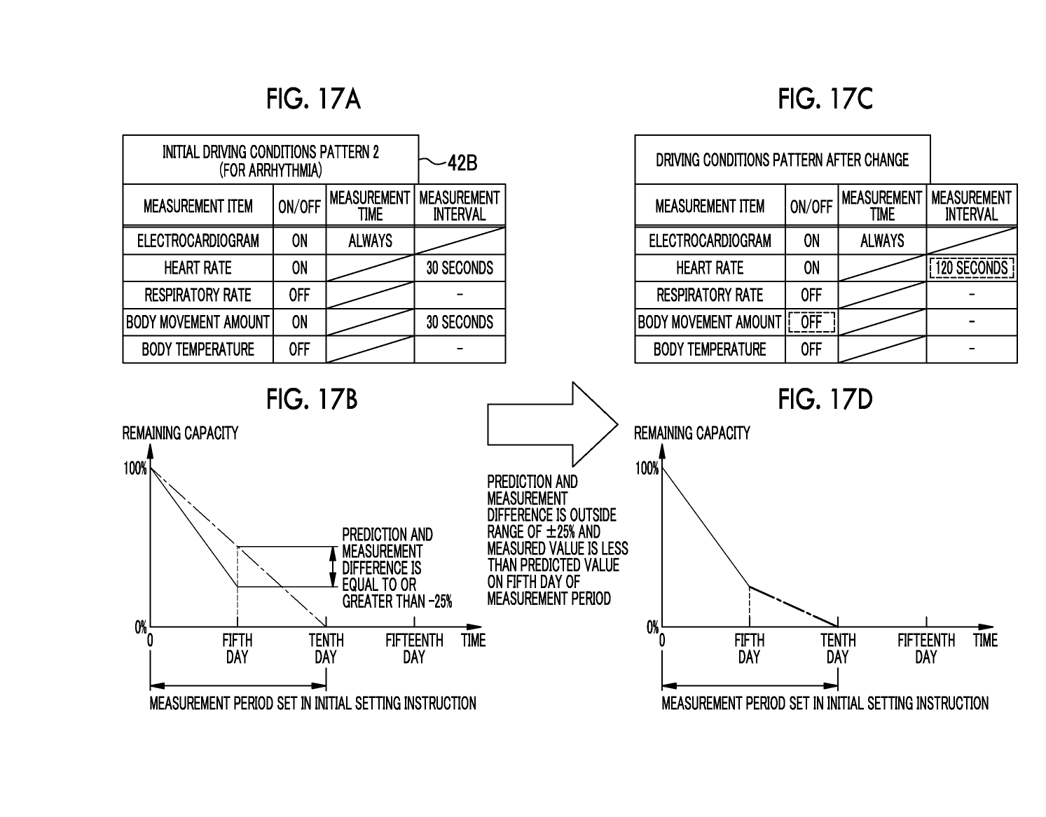

FIGS. 17A to 17D show an example of changing the driving conditions so as to reduce the driving power of the wearable biological sensor device 11 in a case where the measured value of the remaining capacity of the battery 17 is less than the predicted value. FIGS. 17A and 17B on the left side of the arrow show a state before changing the driving conditions, and FIGS. 17C and 17D on the right side of the arrow show a state after changing the driving conditions. Before changing the driving conditions, as shown in FIGS. 17A and 17B, the initial driving conditions 42B of the pattern 2 shown in FIG. 8 are selected and 10 days are set as a measurement period in the initial setting instruction, as in FIGS. 16A and 16B.

As shown in FIG. 17B, for example, in a case where the prediction and measurement difference between the measured value shown by the solid line and the predicted value shown by the one-dot chain line is −25% or more on the fifth day of the measurement period, that is, the prediction and measurement difference is outside the range of ±25% and the measured value is less than the predicted value, the determination unit 56 reduces the number of measurement items by changing the designation of ON/OFF of the body movement amount sensor 27 to OFF from ON, as shown in FIG. 17C. In addition, the measurement interval is increased by changing the measurement interval of the heart rate sensor 25 to 120 seconds from 30 seconds. In this case, as shown in FIG. 17D, the determination unit 56 adjusts the measurement interval and the number of measurement items to be reduced, based on the measured value shown by the solid line and the power consumption information 44, so that the predicted value after the fifth day shown by the one-dot chain line becomes 0% of the set value exactly on the tenth day that is the end date and time.

In the cases shown in FIGS. 16A to 16D and 17A to 17D, the driving conditions are changed so as to reduce the driving power of the wearable biological sensor device 11. Accordingly, in a remaining capacity estimation graph after the change of the driving conditions, the slope is gentle compared with that before the change of the driving conditions.

In FIGS. 14A to 17D, examples of changing the driving conditions from the initial driving conditions 42 once during the measurement period have been mentioned. However, the driving conditions can be changed any number of times during the measurement period whenever the measurement period is changed or whenever the prediction and measurement difference is outside the range of ±25%. In addition, in FIGS. 14A to 17D, the set value of the remaining capacity at the end of the measurement period is 0%. However, the set value may be 5% or 10% so that a slight capacity is left at the end of the measurement period.

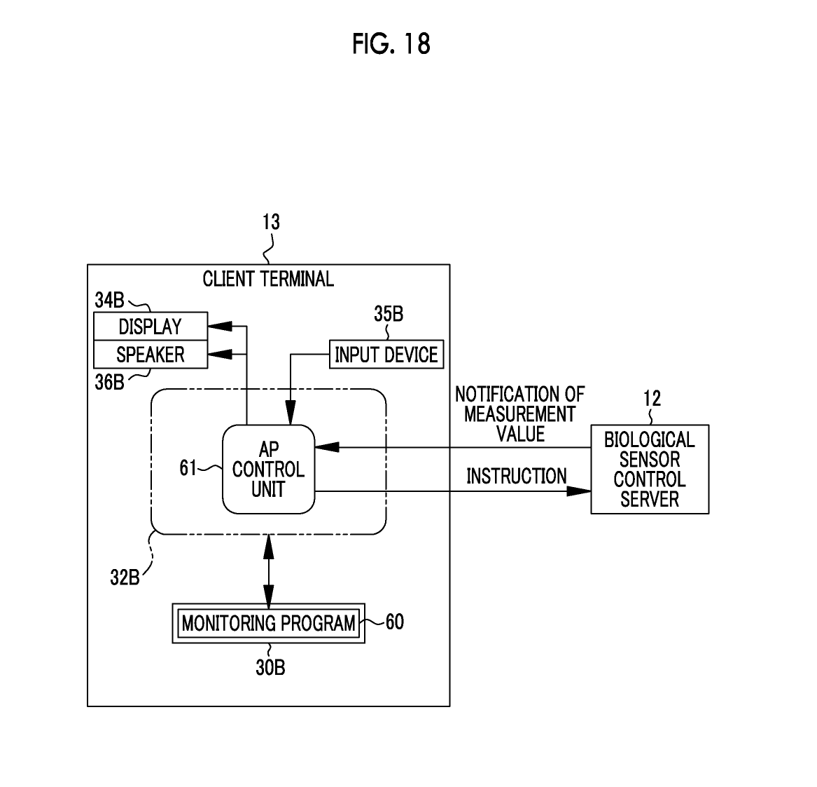

In FIG. 18, a monitoring program 60 as an AP is stored in the storage device 30B of the client terminal 13. When the monitoring program 60 is started, the CPU 32B of the client terminal 13 cooperates with the memory 31 or the like to function as an AP control unit 61.

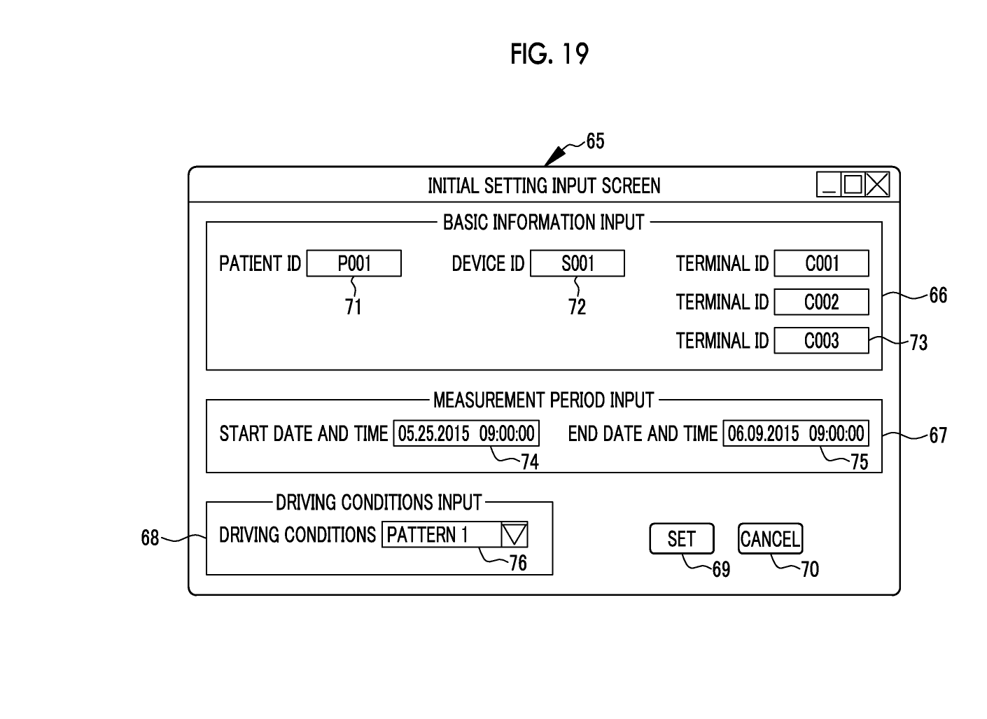

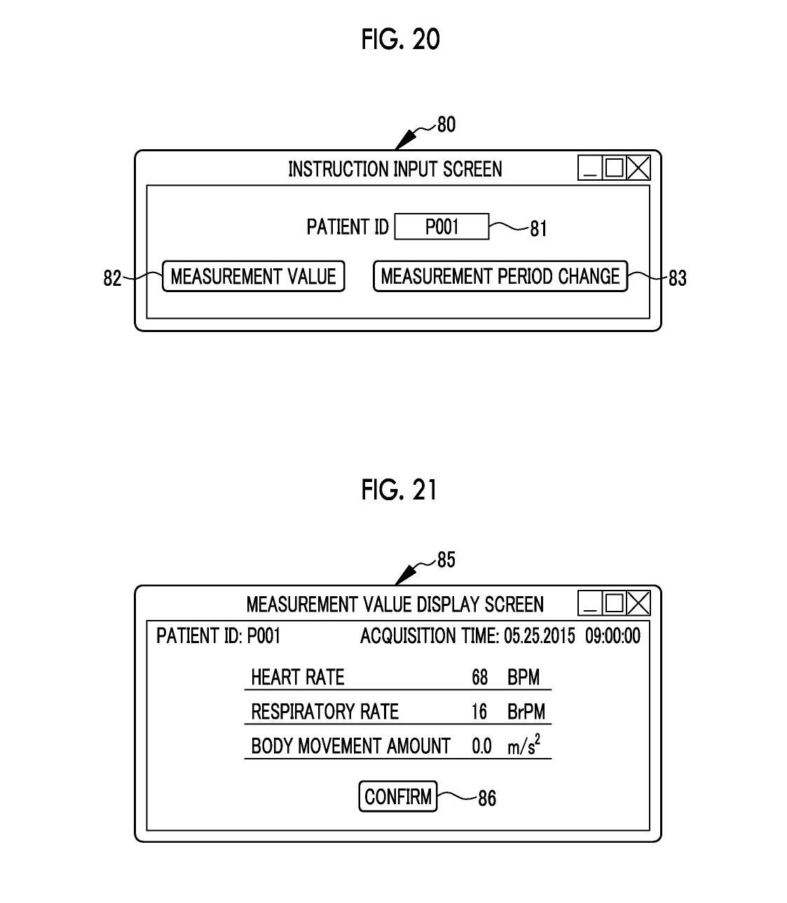

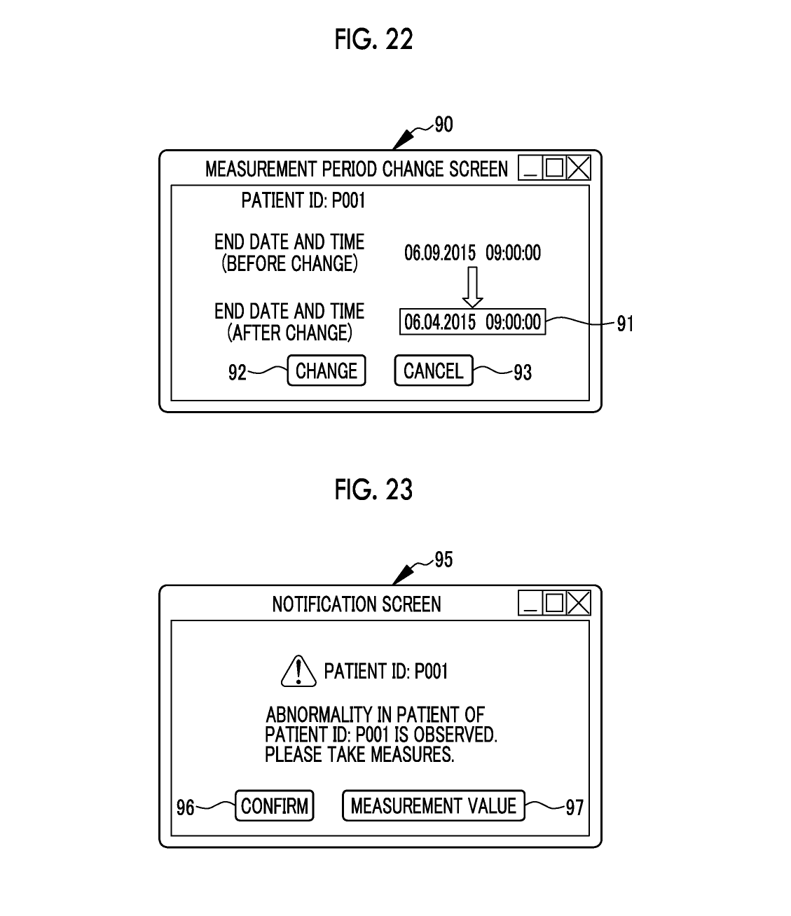

The AP control unit 61 displays various screens for receiving the input of various instructions, specifically, an initial setting input screen 65 for receiving the input of an initial setting instruction (refer to FIG. 19), an instruction input screen 80 for receiving the input of a measurement value request instruction (refer to FIG. 20), and a measurement period change screen 90 for receiving the input of a measurement period change instruction (refer to FIG. 22) on the display 34B, and receives various instructions that are input from the input device 35B through these various screens. The AP control unit 61 transmits various instructions to the biological sensor control server 12.

In addition, the AP control unit 61 displays a measurement value display screen 85 (refer to FIG. 21) on the display 34B based on the measurement value from the information management unit 54. In response to the notification from the abnormality determination unit 55, the AP control unit 61 displays a notification screen 95 (refer to FIG. 23) on the display 34B, and outputs a warning sound, such as a beep, from a speaker 36B. In a case where the client terminal 13 has a vibration function, the AP control unit 61 may operate the vibration function along with the display of the notification screen 95 and the output of the warning sound.

In FIG. 19, the initial setting input screen 65 includes a basic information input region 66, a measurement period input region 67, a driving conditions input region 68, a setting button 69, and a cancel button 70. A patient ID input box 71, a device ID input box 72, and a terminal ID input box 73 are provided in the basic information input region 66. A start date and time input box 74 and an end date and time input box 75 are provided in the measurement period input region 67. A pull-down menu 76 for selecting one type of initial driving conditions 42 among a plurality of types of initial driving conditions 42 is provided in the driving conditions input region 68.

In a case where a desired patient ID, desired start date and time, desired end date and time, and the like are input to the respective input boxes 71 to 75, one type of initial driving conditions 42 are selected in the pull-down menu 76, and the setting button 69 is selected, an initial setting instruction is transmitted to the biological sensor control server 12 from the AP control unit 61.

In FIG. 20, a patient ID input box 81, a measurement value button 82, and a measurement period change button 83 are provided in the instruction input screen 80. In a case where the patient ID of the desired patient P is input to the input box 81 and the measurement value button 82 is selected, a measurement value request instruction is transmitted to the biological sensor control server 12 from the AP control unit 61.

FIG. 21 shows the measurement value display screen 85 that is displayed based on the measurement value transmitted from the information management unit 54 in response to the measurement value request instruction. A patient ID designated on the instruction input screen 80, acquisition time of measurement values, and measurement values are displayed on the measurement value display screen 85. Reference numeral 86 indicates a confirmation button for eliminating the measurement value display screen 85.

FIG. 22 shows the measurement period change screen 90 displayed in a case where the patient ID of the desired patient P is input to the input box 81 and the measurement period change button 83 is selected on the instruction input screen 80. A patient ID designated on the instruction input screen 80 and the end date and time before change corresponding to the patient ID, which is registered in the remaining capacity and period table 43, are displayed on the measurement period change screen 90.

The measurement period change screen 90 includes an input box 91 for inputting the end date and time after change, a change button 92, and a cancel button 93. In a case where desired end date and time is input to the input box 91 and the change button 92 is selected, a measurement period change instruction is transmitted to the biological sensor control server 12 from the AP control unit 61.

In FIG. 23, a message showing that an abnormality is observed in the patient P of a patient ID included in a notification, a confirmation button 96, and a measurement value button 97 are displayed on the notification screen 95. The confirmation button 96 is a button for eliminating the notification screen 95 similar to the confirmation button 86 of the measurement value display screen 85. In addition, the measurement value button 97 is a button having the same function as the measurement value button 82 of the instruction input screen 80, and is a button for displaying the measurement value display screen 85 of the patient P of a patient ID included in a notification.

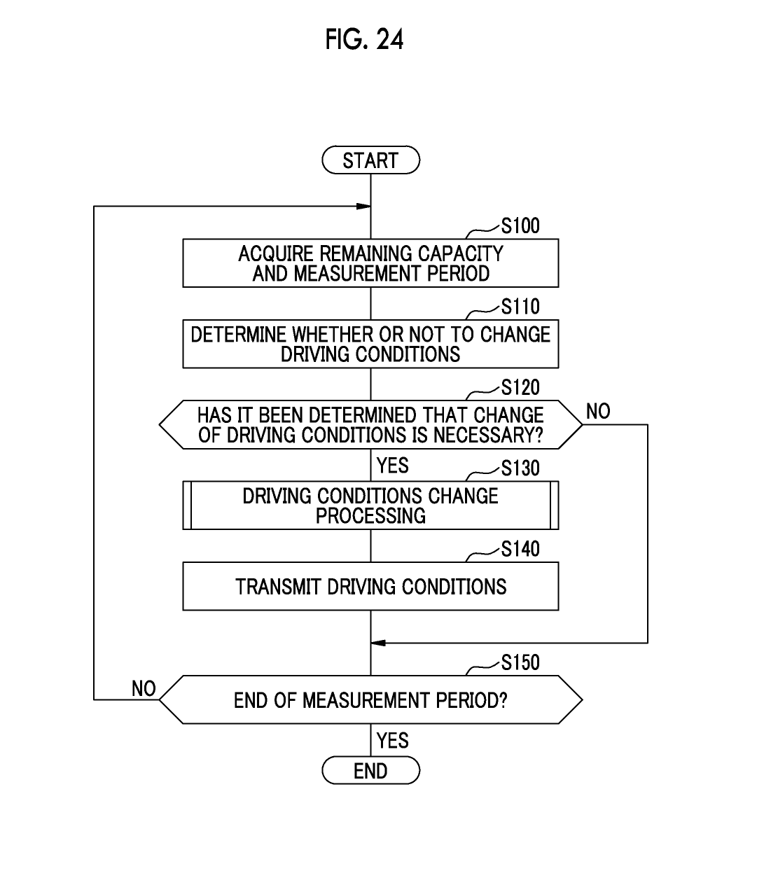

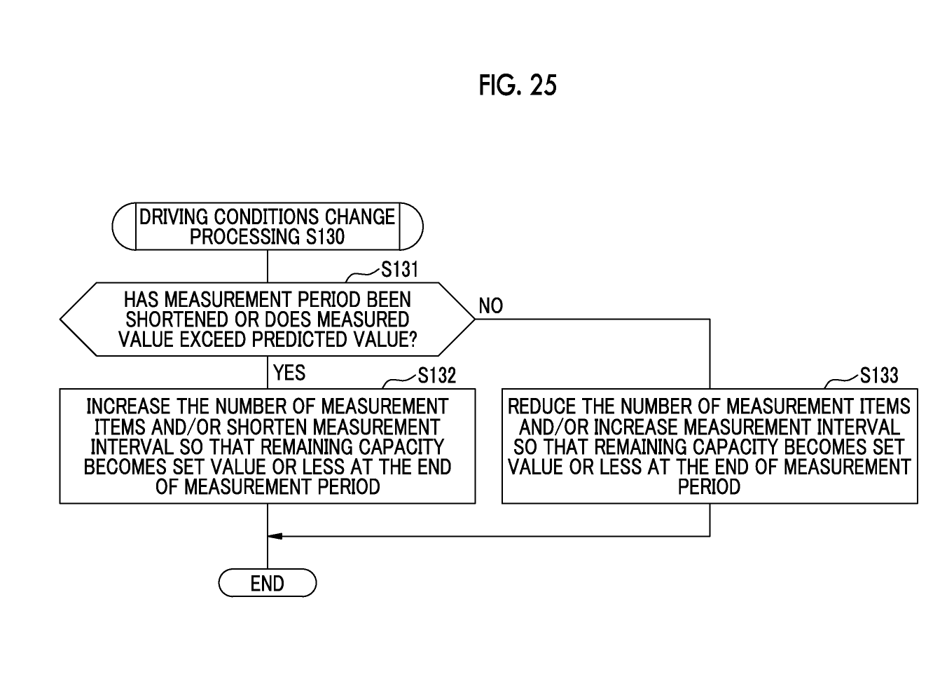

Hereinafter, the operation based on the above configuration will be described with reference to the flowchart shown in FIGS. 24 and 25. First, the wearable biological sensor device 11 is attached to the patient P selected by the doctor D. The doctor D operates the client terminal 13 to start the monitoring program 60, and displays the initial setting input screen 65 on the display 34B. Then, the doctor D inputs and selects a patient ID, a device ID, a terminal ID, a measurement period (start date and time and end date and time), and the initial driving conditions 42 through the initial setting input screen 65, and selects the setting button 69. As a result, an initial setting instruction is transmitted to the biological sensor control server 12 from the client terminal 13.

In the biological sensor control server 12, an initial setting instruction is received by the instruction receiving unit 50, and a measurement period included in the initial setting instruction is acquired by the second acquisition unit 52. In addition, the initial driving conditions 42 selected in the initial setting instruction are transmitted to the wearable biological sensor device 11 from the determination unit 56. In addition, various kinds of information, such as a patient ID included in the initial setting instruction, are registered in the remaining capacity and period table 43 by the information management unit 54.

In the wearable biological sensor device 11, the initial driving conditions 42 from the determination unit 56 are received. Then, the driving of each of the sensors 24 to 28 according to the initial driving conditions 42 is started at the start date and time of the measurement period attached to the initial driving conditions 42. The measurement value of each of the sensors 24 to 28 is transmitted to the biological sensor control server 12.

In the wearable biological sensor device 11, the measurement of the remaining capacity of the battery 17 is started at the start of the driving of each of the sensors 24 to 28, and the measured remaining capacity is transmitted to the biological sensor control server 12.

In the biological sensor control server 12, a measurement value is acquired by the third acquisition unit 53. The measurement value is registered in the measurement value table 41 by the information management unit 54, and is output to the abnormality determination unit 55 from the information management unit 54. Then, the abnormality determination unit 55 determines whether or not each measurement item satisfies the abnormality determination conditions 46.

In a case where the abnormality determination unit 55 determines that each measurement item satisfies the abnormality determination conditions 46, a notification is transmitted to the client terminal 13 from the abnormality determination unit 55. In the client terminal 13, the notification screen 95 corresponding to the notification from the abnormality determination unit 55 is displayed on the display 34B, and a warning sound is output from the speaker 36B. Accordingly, a medical staff member can reliably see that an abnormality has occurred in the condition of the patient P. In addition, the medical staff member can input a measurement value request instruction through the instruction input screen 80, and can display the measurement value display screen 85 of the patient P, about whom the medical staff member is worried personally, on the display 34B.

As shown in step S100 of FIG. 24, in the biological sensor control server 12, a remaining capacity is acquired by the first acquisition unit 51, and a measurement period is acquired by the second acquisition unit 52. Then, based on the remaining capacity and the measurement period, the determination unit 56 determines whether or not to change the driving conditions during the measurement period (step S110). More specifically, the determination unit 56 monitors a change in the measurement period and a prediction and measurement difference during the measurement period, and determines whether or not to change the driving conditions based on the monitoring results.

In a case where there is no change in the measurement period or in a case where the prediction and measurement difference is within a predetermined range, the determination unit 56 determines that the change of the driving conditions is not necessary (NO in step S120). On the other hand, in a case where the measurement period has been changed or in a case where the prediction and measurement difference is outside the range, the determination unit 56 determines that the change of the driving conditions is necessary (YES in step S120). In this case, driving conditions change processing is performed by the determination unit 56 (step S130).

Referring to FIG. 25, in the driving conditions change processing S130, it is determined whether the measurement period has been shortened or the measured value exceeds the predicted value (step S131).

In a case where the measurement period has been shortened or in a case where the measured value exceeds the predicted value (YES in step S131), the determination unit 56 increases the number of measurement items and/or shortens the measurement interval so that the remaining capacity becomes a set value or less at the end of the measurement period (step S132).

On the other hand, in a case where the measurement period has been extended or in a case where the measured value is less than the predicted value (NO in step S131), the determination unit 56 reduces the number of measurement items and/or increases the measurement interval so that the remaining capacity becomes a set value or less at the end of the measurement period (step S133). Then, the driving conditions change processing S130 ends.

As shown in step S140 of FIG. 24, the driving conditions changed in the driving conditions change processing S130 are transmitted to the wearable biological sensor device 11 from the determination unit 56. Each process of S100 to S140 is continued until the measurement period ends (YES in step S150).

In the wearable biological sensor device 11, in a case where the driving conditions after change are received, the driving of each of the sensors 24 to 28 is changed according to the changed driving conditions. For example, driving power from the battery 17 is supplied to a sensor for which the designation of ON/OFF has been changed to ON from OFF, so that the driving of the sensor is started. In addition, a sensor for which the measurement interval has been changed performs measurement at the changed measurement intervals.

Since the driving conditions are determined based on the remaining capacity and the measurement period, it is possible to avoid a situation in which the power of the battery 17 is exhausted before the end of the measurement period so that it is not possible to perform measurement or the remaining capacity of the battery 17 is relatively large at the end of the measurement period. Accordingly, it is possible to effectively use the power of the battery 17 within the measurement period.

It is determined whether or not to change the driving conditions during the measurement period, and the driving conditions are changed immediately in a case where it is determined that the change of the driving conditions is necessary. Accordingly, the wearable biological sensor device 11 can be driven in consistently suitable driving conditions according to the current situation.

A change in the measurement period and the prediction and measurement difference are monitored during the measurement period, and it is determined that the change of the driving conditions is necessary in a case where the prediction and measurement difference is outside a predetermined range. Therefore, it is possible to respond to a case where the measurement period has been changed suddenly for the convenience of the patient P or a case where a difference from the assumed use of environment of the biological sensor device 11 is relatively large.

The driving conditions in which the remaining capacity becomes a set value or less at the end of the measurement period are determined. Accordingly, if the set value is 0% or a value close to 0%, it is possible to realize an ideal use that the power of the battery 17 is exhausted exactly at the end of the measurement period. For this reason, in a case where the battery 17 is a disposable battery, no halfway remaining capacity is left. Therefore, it is possible to effectively use resources. Typically, in the case of the patient P who regularly visits the medical facility 16, the latest measurement values immediately before visiting the medical facility 16 are useful for diagnosis by the doctor D in many cases. Accordingly, it is possible to improve the diagnosis accuracy by performing measurement for as many measurement items as possible until the end of the measurement period and performing measurement continuously.

In a case where the measurement period is shortened or in a case where the measured value exceeds the predicted value, the number of measurement items is increased or the measurement interval is shortened. Accordingly, it is possible to acquire more detailed biological information. If it is possible to obtain more detailed biological information, in a case where the patient P shows the symptoms of a certain disease during the measurement period, the more detailed biological information can be significantly useful for the determination of the disease.

On the other hand, in a case where the measurement period is extended or in a case where the measured value is less than the predicted value, the driving conditions are changed so as to reduce the driving power of the wearable biological sensor device 11 by reducing the number of measurement items or by increasing the measurement interval. Therefore, it is possible to increase the driving time of the wearable biological sensor device 11.

In addition, in a case where a day, on which the remaining capacity in the remaining capacity estimation graph is equal to or greater than the set value, is set to the end date of the measurement period and it is clear on a day before the end date that the remaining capacity will become equal to or greater than the set value at the end of the measurement period (for example, in a case where the end date of the measurement period is set to the fifth day and it is estimated that the remaining capacity will become 50% or more on the fourth day before the end date in a case where the initial driving conditions 42A are selected in which it is estimated that the remaining capacity will become 0% exactly on the fifteenth day as shown in FIGS. 14B and 15B), it is preferable to change the driving conditions by increasing the number of measurement items on the day before the end date or by shortening the measurement interval so that the remaining capacity surely becomes a set value or less than at the end of the measurement period.

The patient P is not limited to a patient recuperating at home 15, and may be a patient admitted to the medical facility 16. In this case, the wearable biological sensor device 11 and the biological sensor control server 12 are communicably connected to each other through a network, such as a local area network (LAN) provided in the medical facility 16. In addition, in this case, for example, the morning of the next day of the admission date is set to the start date and time, and the night of the day before the scheduled discharge date or the scheduled rehabilitation start date is set to the end date and time.

The patient P may be a patient who receives house calls or visiting care periodically at home 15. In this case, for example, “after current house calls or visiting care” is set to the start date and time, and the morning of the next scheduled house calls date or the next scheduled visiting care date is set to the end date and time.

In the first embodiment described above, a measurement period manually set by the medical staff is acquired by the second acquisition unit 52. However, a measurement period may be automatically acquired from a hospital information system (HIS) server or an electronic medical record server that manages an electronic medical record in which the next scheduled examination date of the patient P and the like are described.

The predetermined range of the prediction and measurement difference for determining whether or not to change the driving conditions is set to ±25%. However, this is just an example, and the predetermined range of the prediction and measurement difference may be set to a range of ±10% or may be set to a range of ±50%. Instead of setting the predetermined range to the same value uniformly, the predetermined range of the prediction and measurement difference may be changed depending on the measurement period. For example, the first half of the measurement period may be set to a range of ±50% and the second half of the measurement period may be set to a range of ±10%. In addition, instead of monitoring the prediction and measurement difference over the entire measurement period, a day to monitor the prediction and measurement difference may be limited to, for example, a day before the end date, and it may be determined whether or not to change the driving conditions based on the monitoring results.

Instead of or in addition to increasing or reducing the number of measurement items or increasing or shortening the measurement interval, the measurement time may be increased or shortened. For example, in a case where the wearable biological sensor device 11 is driven under the initial driving conditions 42B shown in FIG. 8 and the measurement period is increased, the measurement time of the electrocardiogram sensor 24 is changed to 09:00:00 to 12:00:00 from always. Alternatively, the measurement time of each of the sensors 25 to 28 other than the electrocardiogram sensor 24 may be set to be able to be designated, so that the measurement time of each of the sensors 25 to 28 other than the electrocardiogram sensor 24 is changeable.

Essential measurement items may be set in advance for every initial driving conditions 42, and the designation of OFF may be made to be invalid only for the essential measurement items in the case of changing the driving conditions so as to reduce the driving power of the wearable biological sensor device 11 by reducing the number of measurement items. For example, in the case of the initial driving conditions 42A for patients of the chronic obstructive pulmonary disease shown in FIG. 7, the respiratory rate and the body movement amount are set as essential measurement items. In this manner, it is possible to prevent the erroneous designation of OFF for a measurement item that needs to be intensively monitored.

Instead of or in addition to the exemplified sensors 24 to 28, a sensor for measuring the skin impedance of the patient P as biological information or a sensor for measuring the blood oxygen concentration of the patient P may be provided in the sensor unit 20.

Although the wearable biological sensor device 11 is exemplified in which the sensors 24 to 28 are integrally provided, the sensors 24 to 28 may be separately provided, and each sensor may be a wearable biological sensor device including the battery 17, the wireless transmission and reception unit 21, or the like. In this case, the first acquisition unit 51 acquires the remaining capacity from each wearable biological sensor device, and the determination unit 56 determines the driving conditions of each wearable biological sensor device.

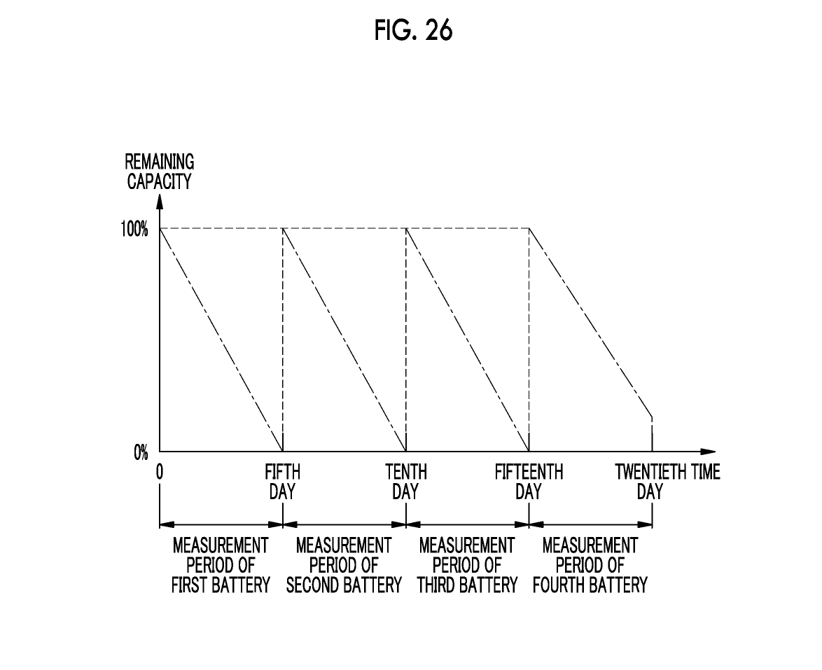

In addition, although a case where there is one battery 17 has been described, a plurality of batteries 17 may be prepared, and the patient P may perform replacement therebetween. The remaining capacity estimation graph of the initial driving conditions 42 in this case is obtained by connecting remaining capacity estimation graphs of the individual batteries 17 to each other in the time axis direction, for example, as shown in FIG. 26 in a case where four batteries 17 are prepared. In this case, the determination unit 56 acquires information indicating which battery 17 is being used currently, and determines the driving conditions in consideration of the information. For example, in a case where four batteries 17 are prepared, the third battery 17 is being used currently, and it is determined that the remaining capacity is likely to be left at the end of the measurement period by reducing the number of measurement items or shortening the measurement interval at the third battery 17, the driving conditions are changed so as to use the fourth battery 17 for the next measurement.

The battery 17 may be a chargeable secondary battery without being limited to the disposable battery. Even in a case where the battery 17 is a secondary battery, a situation in which charging is performed regardless of the remaining capacity so that the battery 17 is deteriorated can be prevented by adopting the ideal use in which the power of the battery 17 is exhausted exactly at the end of the measurement period.

### Second Embodiment

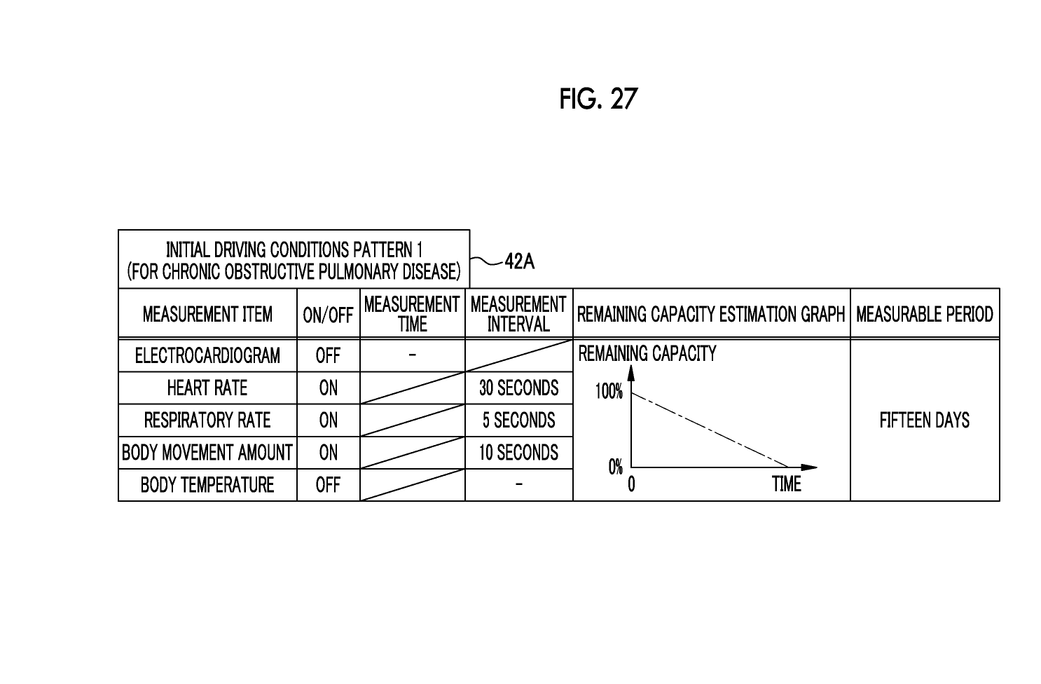

According to the remaining capacity estimation graph of the initial driving conditions 42, in the case of performing measurement under the initial driving conditions 42, it is possible to see how long it will take for the remaining capacity to become 0% from 100%. That is, it is possible to see the approximate measurement allowed period. If the measurement period set on the initial setting input screen 65 by the doctor D falls within the measurement allowed period, a probability that a situation, in which the power of the battery 17 is exhausted before the end of the measurement period so that it is not possible to perform measurement, will be avoided is high. Therefore, in the second embodiment shown in FIGS. 27 to 29, in a case where the measurement period acquired by the second acquisition unit 52 does not fall within the estimated measurement allowed period, a notification indicating that the measurement period exceeds the measurement allowed period is sent to the client terminal 13 from the determination unit 56.

In this case, in the initial driving conditions 42, not only the remaining capacity estimation graph but also the measurement allowed period is registered, as in the initial driving conditions 42A exemplified in FIG. 27.

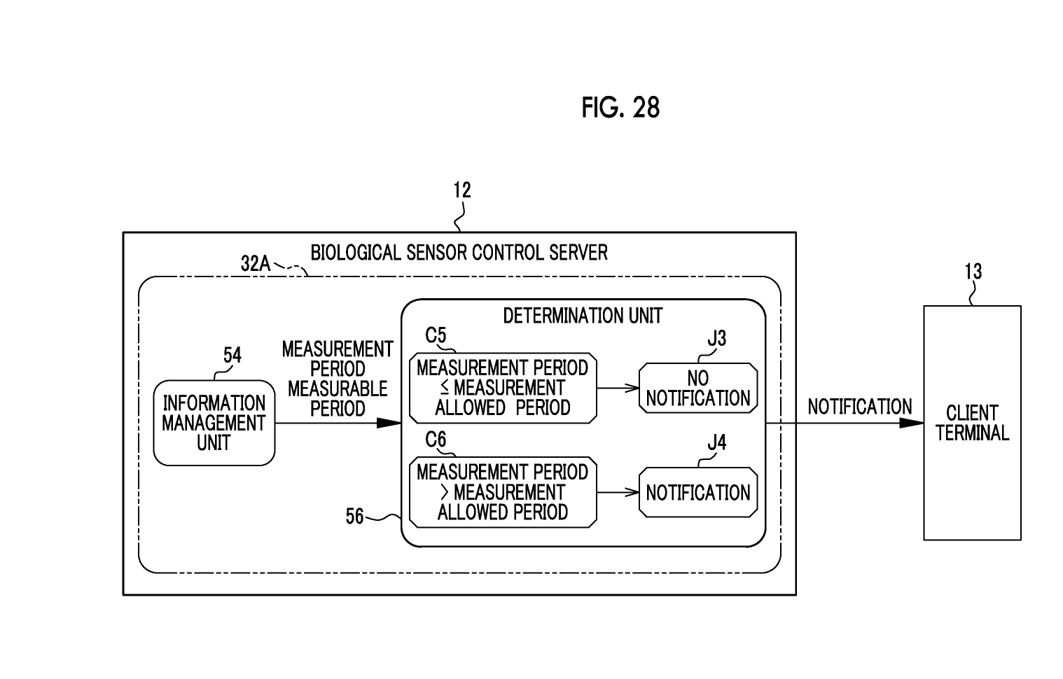

In addition, as shown in FIG. 28, when the initial setting instruction is received by the instruction receiving unit 50, the determination unit 56 receives the measurement allowed period registered in the initial driving conditions 42 and the measurement period, which is acquired by the second acquisition unit 52 and is registered in the remaining capacity and period table 43, from the information management unit 54. Then, the measurement allowed period and the measurement period that have been received are compared with each other.

In a case where the measurement period is equal to or less than the measurement allowed period, that is, in a case where the measurement period falls within the measurement allowed period (C5), the determination unit 56 does nothing (J3). On the other hand, in a case where the measurement period is longer than the measurement allowed period, that is, in a case where the measurement period does not fall within the measurement allowed period (C6), the determination unit 56 transmits a notification indicating that the measurement period exceeds the measurement allowed period to the client terminal 13 that is a transmission source of the initial setting instruction (J4).

For example, in a case where the initial driving conditions 42A, in which the measurement allowed period is 15 days, are selected in the initial setting instruction and 10 days are set as a measurement period, no notification is transmitted from the determination unit 56 since the measurement period falls within the measurement allowed period. On the other hand, in a case where 20 days are set as a measurement period, a notification is transmitted to the client terminal 13 from the determination unit 56 since the measurement period does not fall within the measurement allowed period. In FIG. 28, functional units other than the information management unit 54 and the determination unit 56 are omitted.

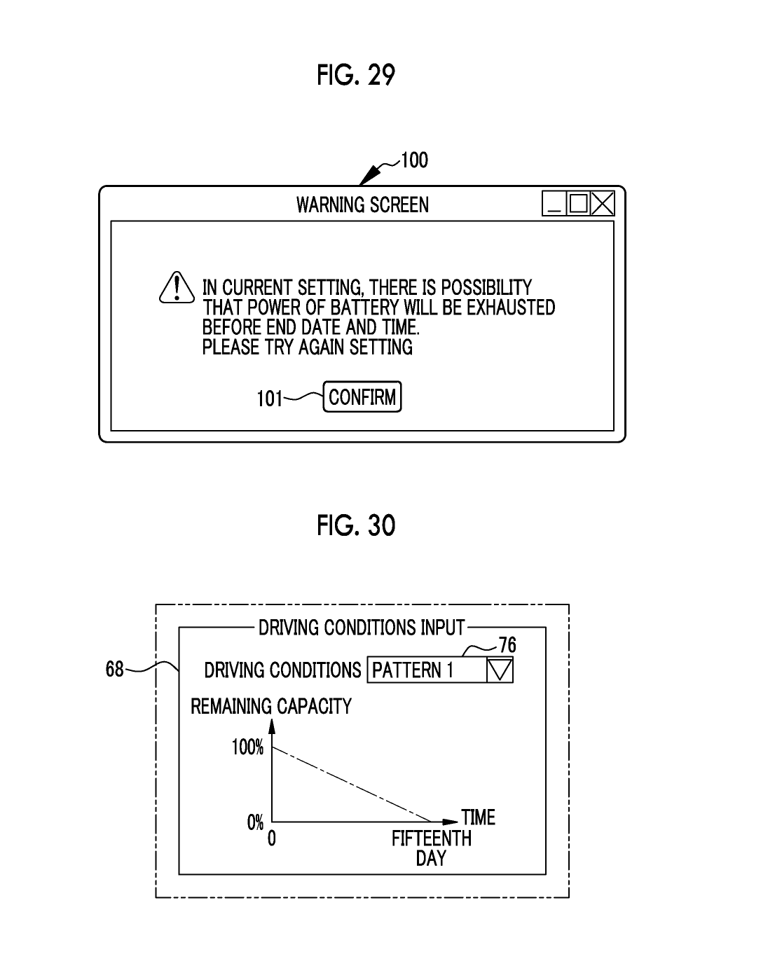

The AP control unit 61 displays a warning screen 100 shown in FIG. 29 on the display 34B in response to the notification indicating that the measurement period from the determination unit 56 exceeds the measurement allowed period. For example, the warning screen 100 is popup-displayed on the initial setting input screen 65. A message showing that the measurement period exceeds the measurement allowed period and the power of the battery 17 may be exhausted before the end date and time in this setting, a message prompting the redoing of setting, and a confirmation button 101 for eliminating the warning screen 100 are displayed on the warning screen 100.

Thus, since a notification indicating that the measurement period exceeds the measurement allowed period is transmitted to the client terminal 13 from the determination unit 56 in a case where the measurement period does not fall within the measurement allowed period, it is possible to further increase the probability that a situation, in which the power of the battery 17 is exhausted before the end of the measurement period so that it is not possible to perform measurement, will be avoided.

In addition, as a measure to avoid the situation in which the power of the battery 17 is exhausted before the end of the measurement period so that it is not possible to perform measurement, for example, as shown in FIG. 30, a method of displaying the measurement allowed period and the remaining capacity estimation graph of the initial driving conditions 42, which are selected in the pull-down menu 76, in the driving conditions input region 68 of the initial setting input screen 65 may be adopted.

Before transmitting the initial driving conditions 42 or the driving conditions changed by the determination unit 56 to the wearable biological sensor device 11, a medical staff member may manually correct the initial driving conditions 42 or the driving conditions changed by the determination unit 56. In this case, the AP control unit 61 displays a correction screen for receiving the input of an instruction to correct the driving conditions on the display 34B, and the instruction receiving unit 50 receives the correction instruction as a manual setting instruction.

In this case, a remaining capacity estimation graph after correction may be created with reference to the power consumption information 44, and the remaining capacity estimation graph after correction and the remaining capacity estimation graph before correction may be displayed on the display 34B so as to be able to be compared with each other.

By applying the second embodiment described above, a measurement allowed period in the case of performing measurement under the driving conditions after correction may be estimated with reference to the power consumption information 44 and the remaining capacity acquired by the first acquisition unit 51. In a case where the measurement period does not fall within the estimated measurement allowed period, a notification indicating that the measurement period exceeds the measurement allowed period may be transmitted from the determination unit 56 to the client terminal 13 that is a transmission source of the correction instruction.

The driving conditions changed by the determination unit 56 may be displayed for the medical staff, so that the driving conditions are transmitted to the wearable biological sensor device 11 after obtaining the approval of the medical staff. There is no particular problem in the case of changing the driving conditions so as to increase the number of measurement items. However, in the case of changing the driving conditions so as to reduce the number of measurement items, diagnosis may be affected if essential measurement items, such as the respiratory rate and the body movement amount of the initial driving conditions 42A for patients of the chronic obstructive pulmonary disease described above, are reduced. For this reason, it is preferable to adopt a configuration of obtaining the approval of the medical staff in the case of changing the driving conditions at least so as to reduce the number of measurement items. In addition, an instruction to correct the driving conditions may be received at the time of approval.

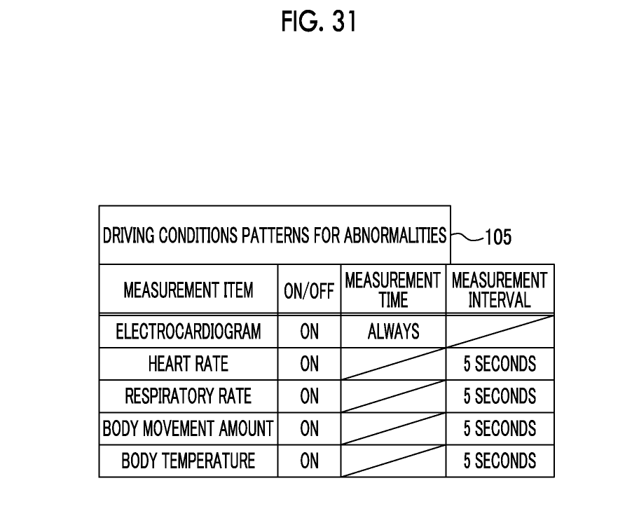

In the first embodiment described above, as processing in a case where the abnormality determination unit 55 determines that the measurement item satisfies the abnormality determination conditions 46, only the processing of transmitting a notification, which indicates that it has been determined that the measurement item satisfies the abnormality determination conditions 46, to the client terminal 13 has been mentioned. However, in addition to this, in a case where the abnormality determination unit 55 determines that the measurement item satisfies the abnormality determination conditions 46, driving conditions for abnormalities 105 shown in FIG. 31 may be set as the driving conditions regardless of the remaining capacity and the measurement period.

Referring to FIG. 31, in the driving conditions for abnormalities 105, ON is designated for all of the sensors 24 to 28, “always” is designated as the measurement time of the electrocardiogram sensor 24, and 5 seconds is designated as the measurement interval of each of the sensors 25 to 28 other than the electrocardiogram sensor 24. That is, the driving conditions for abnormalities 105 are driving conditions for performing measurement for all of the measurement items at relatively short measurement intervals.

Thus, in a case where the abnormality determination unit 55 determines that the measurement item satisfies the abnormality determination conditions 46, if the driving conditions are changed to the driving conditions for abnormalities 105, it is possible to acquire more detailed biological information when a certain abnormality has occurred in the condition of the patient P. This can be useful for the determination of the disease.

In the driving conditions for abnormalities 105, power consumption is large since measurement is performed at relatively short measurement intervals for all of the measurement items. Accordingly, if the wearable biological sensor device 11 is driven for a long time under the driving conditions for abnormalities 105, a probability that the power of the battery 17 will be exhausted before the end date and time is high. Therefore, in a case where a state, in which the abnormality determination unit 55 determines that the measurement item does not satisfy the abnormality determination conditions 46, continues for a predetermined period after changing the driving conditions to the driving conditions for abnormalities 105, returning to the original driving conditions from the driving conditions for abnormalities 105 is preferable.

When returning to the original driving conditions from the driving conditions for abnormalities 105, a possibility that the prediction and measurement difference is outside a predetermined range and the measured value is less than the predicted value due to rapid power consumption under the driving conditions for abnormalities 105 is high. However, since the determination unit 56 changes the driving conditions so as to reduce the driving power of the wearable biological sensor device 11 so that the remaining capacity becomes a preset value or less at the end of the measurement period, there is no problem.

In the case of using the driving conditions for abnormalities 105, in order to avoid a situation, in which the power of the battery 17 is exhausted before the end date and time, by predicting the power consumption under the driving conditions for abnormalities 105, setting the measurement period to be short can be considered. Then, in a case where the driving conditions are not switched to the driving conditions for abnormalities 105 during the measurement period, a possibility that the remaining capacity will become equal to or greater than the set value at the end of the measurement period is high. In this case, however, since the determination unit 56 determines the driving conditions so that the remaining capacity becomes a set value or less at the end of the measurement period, there is no problem.

The hardware configuration of a computer, which forms the biological sensor control server 12 corresponding to the biological sensor control device of the invention, can be modified in various ways. For example, in order to improve the processing capacity or reliability, the biological sensor control server 12 may be formed by a plurality of server computers that are separated from each other as hardware. For example, the functions of the instruction receiving unit 50, the first acquisition unit 51, the second acquisition unit 52, and the third acquisition unit 53, the function of the information management unit 54, and the functions of the abnormality determination unit 55 and the determination unit 56 may be distributed in three server computers. In this case, the three server computers configure the biological sensor control device. The functions of the first acquisition unit 51, the second acquisition unit 52, and the third acquisition unit 53 may be integrated into one acquisition unit.

In each of the embodiments described above, a case has been exemplified in which a measurement value or a notification is transmitted to the client terminal 13 from the biological sensor control server 12 and the AP control unit 61 of the client terminal 13 generates the measurement value display screen 85, the notification screen 95, or the warning screen 100 and displays the generated screen on the display 34B. However, various screens may be generated on the biological sensor control server 12 side and the screen data may be transmitted to the client terminal 13, and the AP control unit 61 may reproduce the various screens based on the screen data and display the various screens on the display 34B. As screen data, for example, it is possible to use screen data for web distribution that is created by data description language, such as Extensible Markup Language (XML) or (JAVASCRIPT (registered trademark) Object Notation (JSON).

Each functional unit constructed in the CPU 32A of the biological sensor control server 12 may be constructed in the CPU 32B of the client terminal 13, so that the client terminal 13 operates as a biological sensor control device. In this case, the instruction receiving unit 50 receives various instructions directly from the input device 35B. In addition, a functional unit, such as the determination unit 56, may be provided in the wearable biological sensor device 11.

Thus, the hardware configuration of a computer can be appropriately changed according to the required performance, such as processing capacity, safety, or reliability. Needless to say, in order to ensure the safety or reliability, an application program, such as the operation program 40, may be duplicated or may be stored in a plurality of storage devices in a distributed manner, without being limited to hardware.

In each of the embodiments described above, a case has been described in which the biological sensor control server 12 is used in one medical facility 16. However, the biological sensor control server 12 may be configured to be able to be used in a plurality of medical facilities.

In each of the embodiments described above, the biological sensor control server 12 is installed in one medical facility 16, acquires various measurement values by controlling the wearable biological sensor device 11 attached to the patient P who regularly visits the medical facility 16, and provides various kinds of information corresponding to various instructions from the client terminal 13 held by the medical staff member of the medical facility 16.

In order to be available in a plurality of medical facilities, the biological sensor control server 12 is communicably connected to the wearable biological sensor device 11, which is attached to the patient P who regularly visits the plurality of medical facilities, and the client terminals 13, which are held by the medical staff of the plurality of medical facilities, through the network 14. Then, the driving conditions are transmitted from the biological sensor control server 12 to the wearable biological sensor device 11, which is attached to the patient P who regularly visits the plurality of medical facilities, through the network 14, and the measurement values and the remaining capacity from the wearable biological sensor device 11 and various instructions from the client terminals 13, which are held by the medical staff of the plurality of medical facilities, are received by the biological sensor control server 12 through the network 14, and the various kinds of information are provided to the client terminal 13.

In this case, the measurement value table 41, the remaining capacity and period table 43, and the like are managed for each of the plurality of medical facilities. In this case, the installation location and management entity of the biological sensor control server 12 may be a data center managed by a company that is different from the medical facilities, or may be one of the plurality of medical facilities, for example.

The installation location of the biological sensor control server 12 is not limited to the medical facility 16. The biological sensor control server 12 may be installed at home 15 of the patient P. In a case where the biological sensor control server 12 is installed at home 15 of the patient P, it is possible to reduce the amount of communication of information between the home 15 of the patient P and the medical facility 16 through the network 14 compared with a case where the biological sensor control server 12 is installed in the medical facility 16. It is expected that the communication load in the network 14 will be increased due to an increase in the amount of big data according to the progress of the Internet of Things (IoT). Therefore, since the amount of communication through the network 14 is reduced if the biological sensor control server 12 is installed at home 15 of the patient P, it is possible to reduce the communication load of the network 14.

In the invention, it is also possible to appropriately combine the above-described various embodiments or various modification examples. Without being limited to the embodiments described above, it is needless to say that various configurations can be adopted without departing from the scope of the invention. In addition to the program, the invention also extends to a storage medium that stores the program.

What is claimed is:

1. A biological sensor control device for controlling a wearable biological sensor device that is attached to a patient and that performs measurement for measurement items regarding biological information of the patient with power supplied from a built-in battery, comprising:
a first acquisition unit that acquires a remaining capacity of the battery;
a second acquisition unit that acquires a measurement period of the wearable biological sensor device; and
a determination unit that determines driving conditions of the wearable biological sensor device based on the remaining capacity and the measurement period.

2. The biological sensor control device according to claim 1,
wherein the determination unit determines the driving conditions such that the remaining capacity becomes a preset value or less at the end of the measurement period.

3. The biological sensor control device according to claim 1,
wherein the determination unit determines whether or not to change the driving conditions during the measurement period.

4. The biological sensor control device according to claim 2,
wherein the determination unit determines whether or not to change the driving conditions during the measurement period.

5. The biological sensor control device according to claim 3,
wherein the determination unit monitors a change in the measurement period and a prediction and measurement difference, which is a difference between a predicted value and a measured value of the remaining capacity, during the measurement period, and determines whether or not to change the driving conditions based on monitoring results.

6. The biological sensor control device according to claim 4,
wherein the determination unit monitors a change in the measurement period and a prediction and measurement difference, which is a difference between a predicted value and a measured value of the remaining capacity, during the measurement period, and determines whether or not to change the driving conditions based on monitoring results.

7. The biological sensor control device according to claim 5,
wherein the determination unit determines that the driving conditions are to be changed in a case where the measurement period has been changed or in a case where the prediction and measurement difference is outside a predetermined range.

8. The biological sensor control device according to claim 6,
wherein the determination unit determines that the driving conditions are to be changed in a case where the measurement period has been changed or in a case where the prediction and measurement difference is outside a predetermined range.

9. The biological sensor control device according to claim 7,
wherein the determination unit changes the driving conditions so as to increase driving power of the wearable biological sensor device in a case where the measurement period is shortened or in a case where the measured value exceeds the predicted value.

10. The biological sensor control device according to claim 8,
wherein the determination unit changes the driving conditions so as to increase driving power of the wearable biological sensor device in a case where the measurement period is shortened or in a case where the measured value exceeds the predicted value.

11. The biological sensor control device according to claim 1,
wherein the wearable biological sensor device performs measurement for the plurality of measurement items, and
the determination unit determines the number of measurement items, for which measurement is to be performed, as the driving conditions.

12. The biological sensor control device according to claim 2,
wherein the wearable biological sensor device performs measurement for the plurality of measurement items, and
the determination unit determines the number of measurement items, for which measurement is to be performed, as the driving conditions.

13. The biological sensor control device according to claim 9,
wherein the wearable biological sensor device performs measurement for the plurality of measurement items, and
the determination unit determines the number of measurement items, for which measurement is to be performed, as the driving conditions.
wherein the determination unit increases the number of measurement items.

14. The biological sensor control device according to claim 1,
wherein the determination unit determines a measurement interval of each of the measurement items as the driving conditions.

15. The biological sensor control device according to claim 2,
wherein the determination unit determines a measurement interval of each of the measurement items as the driving conditions.

16. The biological sensor control device according to claim 9,
wherein the determination unit determines a measurement interval of each of the measurement items as the driving conditions.
wherein the determination unit shortens the measurement interval.

17. The biological sensor control device according to claim 1, further comprising:
an instruction receiving unit that receives a manual setting instruction of the driving conditions,
wherein, in a case where the measurement period acquired by the second acquisition unit does not fall within a measurement allowed period estimated in a case where measurement has been performed under the driving conditions received by the instruction receiving unit, the determination unit sends a notification indicating that the measurement period exceeds the measurement allowed period.

18. An operation method of a biological sensor control device for controlling a wearable biological sensor device that is attached to a patient and that performs measurement for measurement items regarding biological information of the patient with power supplied from a built-in battery, the operation method comprising:
a first acquisition step of acquiring a remaining capacity of the battery;
a second acquisition step of acquiring a measurement period of the wearable biological sensor device; and
a determination step of determining driving conditions of the wearable biological sensor device based on the remaining capacity and the measurement period.

19. Non-transitory computer readable recording medium storing an operation program of a biological sensor control device for controlling a wearable biological sensor device that is attached to a patient and that performs measurement for measurement items regarding biological information of the patient with power supplied from a built-in battery, the operation program causing a computer to execute:
a first acquisition function of acquiring a remaining capacity of the battery;
a second acquisition function of acquiring a measurement period of the wearable biological sensor device; and
a determination function of determining driving conditions of the wearable biological sensor device based on the remaining capacity and the measurement period.

20. A biological sensor system, comprising:
a wearable biological sensor device that is attached to a patient and that performs measurement for measurement items regarding biological information of the patient with power supplied from a built-in battery; and
a biological sensor control device that controls the wearable biological sensor device,
wherein the biological sensor control device comprises a first acquisition unit that acquires a remaining capacity of the battery, a second acquisition unit that acquires a measurement period of the wearable biological sensor device, and a determination unit that determines driving conditions of the wearable biological sensor device based on the remaining capacity and the measurement period.
